(19) United States
(12) Patent Application Publication
(10) Pub. No.: US 2012/0128709 A1
(43) Pub. Date: May 24, 2012

(54) EXPRESSION OF RECOMBINANT PROTEINS

(76) Inventors: Erwin Swennen, Bologna (IT); Salvatore Nocadello, Siena (IT)

(21) Appl. No.: 13/322,015

(22) PCT Filed: May 28, 2010
(86) PCT No.: PCT/IB10/01457

§ 371 Date: Feb. 10, 2012

Related U.S. Application Data
(60) Provisional application No. 61/185,613, filed on Jun. 10, 2009.

(30) Foreign Application Priority Data
May 28, 2009 (IT) MI2009A000946

(51) Int. Cl. A61K 39/108; C07K 14/28; C12N 15/31; C07K 14/245; C12N 15/70; C12P 21/02; A61P 37/04; A61P 31/04; C12N 1/21; C12N 15/63

(52) U.S. Cl. 424/190.1; 435/252.33; 530/350; 536/23.7; 435/320.1; 435/69.3

(57) ABSTRACT

The invention provides auto-inducible systems for expressing recombinant proteins of interest which take advantage of elements of quorum sensing (QS) systems of certain bacteria. These systems can be used to produce commercial quantities of proteins such as antigens, which can be used to prepare pharmaceutical compositions.

The present invention relates generally to the production of recombinant proteins. In particular, the invention relates to the production of recombinant proteins in an auto-induction activation system.

### BACKGROUND OF THE INVENTION

The use of recombinant gene expression for industrial protein production since the early 1970s has become a multi-billion industry. Proteins can be produced recombinantly by making use of expression machinery derived from a host cell. Although protein expression can occur in vitro, host cells are commonly used as factories for recombinant gene expression. Examples of suitable hosts include bacteria, such as the gram-negative bacterium Escherichia coli, animal host cells (e.g., mammalian or insect host cells) and yeasts. However, in spite of the extensive knowledge on the genetics and molecular biology of E. coli and other host cells, obtaining efficient protein production (for example, on an industrial scale) can be difficult, and there remains a need to provide expression systems which can regulate and control protein expression such that the recombinant protein is produced at the right time during host cell culture.

After many years of intensive research some empirical “rules” that can guide the design of expression systems in E. coli have emerged. Among these, tight regulation of promoter activity can allow for a fast initial period of cell growth to high density. Once an optimal density was obtained, protein expression can be triggered through inducible activation of the promoter, using an inducer (a system known as inducible expression). However, there are problems associated with this approach. The cost of inducers, particularly when used on an industrial scale, can be very high. Furthermore, some inducible promoters may affect cell growth and may be incompatible with the subsequent use of the expressed proteins in humans or animals. In addition to the above, use of an inducible promoter requires the active addition of the inducer to the bacterial culture, increasing the chance of contaminating the culture and adding a further component which needs to be removed during subsequent purification of the recombinant protein.

The use of auto-induction (or self-induction) expression systems for recombinant protein production was considered as a solution for eliminating the need to monitor cell growth and add actively the inducer during the growth. In these systems, auto-induction can be brought about, for example, by metabolic changes during growth of the host cell. Auto-induction systems based respectively on use of regulatory elements of the lac operon (e.g., T7lac promoter) and diauxic growth have been described in US2004/0180426. In the diauxic system described in US2004/0180426, the medium contains glucose, glycerol and lactose. Glucose is used as the carbon source during the growth phase and at the same time acts as a catabolic repressor of the T7lac promoter. When glucose is exhausted, glycerol is used as the carbon source. Furthermore, in the absence of glucose, lactose uptake is triggered and the lactose entering the host cell serves to activate the T7lac promoter, thereby stimulating gene expression. A similar system makes use of glucose and proprionate and autoinduction is triggered through proprionate induced activation of the propionate-inducible E. coli prpBCDE promoter, previously described (Lee S K and Keasling J D. Protein Expr Purif. 2008 61:197-203).

However the above-described auto-induction systems rely on exogenous sources of autoinducer (i.e., lactose and propionate respectively) which need to be added at the beginning of the host cell culture. There remains a need to develop an effective, autonomous system of auto-induction which does not rely on the addition of an autoinducer. Furthermore, there remains a need to effectively control gene expression until the host cell culture has reached a level which will allow for maximum efficiency of protein production.

The Quorum Sensing (QS) system is a natural system based on a form of cell-cell communication. QS system was first described in the 1970s for the marine bacterium Vibrio fischeri. It is widespread among bacteria. Bacteria having a QS system can sense the density of their population. QS system is based on the release of an autoinducer by the cells in the medium. Cells respond to threshold concentrations of the autoinducer which can be reached only at a certain cell density (a “quorum”). Once this threshold concentration is reached, a cascade of signal transduction events is activated which results in the activation of target genes under the control of the QS machinery. This system can also allow for the amplification of the autoinducer itself in a positive feedback loop, by a mechanism of auto-regulation. Three types of QS system have been until now described in Gram negative bacteria and/or Gram positive bacteria based on the nature of the autoinducer (Types I to III). Type I was found so far only in Gram negative bacteria and uses acyl homoserine lactone as the autoinducer. One Type I system that was described in detail is the QS system from Vibrio fischeri (Kaplan and Greenberg, 1985 J bacteriol., 163:1210-1214).

Luminescent genes of V. fischeri are activated by QS system in a positive feedback regulation. The lux genes are transcribed by two divergent operons; the left operon contains the luxR gene which encodes the regulatory protein LuxR, and the right operon contains at least 6 genes (luxICDABE). The two operons are separated by a common regulatory region. The gene luxI encodes an autoinducer synthase (LuxI) which produces the autoinducer known as N-(3-oxohexanoyl)-homoserine lactone (HSL). LuxR binds to HSL and the complex acts as an autoinducer complex, LuxR/HSL, which binds to an inducible promoter (lux Box in luxI promotor, P_{luxI}) located upstream of the luxICDABE operon. Binding of LuxR/HSL to this promoter activates the transcription of genes involved in the synthesis of luciferase as well activating transcription of luxI, thereby functioning to increase production of HSL in a positive feedback loop. LuxR/HSL also binds to the luxR promoter which regulates its synthesis (March and Bentley, Curr Opin Biotechnol. 2004 15:495-502). The genes of the natural QS system have been expressed in E. coli, and its components were used to demonstrate cell-cell communication in this host (Engebrecht, Cell 1983, 32: 773-781; Devine et al., Proc Natl Acad Sci USA. 1989 86:5688-92).

Some regulatory elements of the QS system from V. fischeri have been used for recombinant production of certain proteins in an inducible expression system. U.S. Pat. No. 5,196,318, reports an expression system which contains an intact luxR gene and a gene of interest operably linked to the luxI promoter. Expression of the gene of interest is switched on by addition of exogenous autoinducer (HSL).

One problem with the QS system described above is that it requires the addition of an exogenous autoinducer. There remains a need to provide a system of autoinduction which is independent of such additions to the host cell culture and which autonomously triggers gene expression at the desired level and during the right phase of cell culture.

### SUMMARY OF THE INVENTION

The invention provides isolated mutant LuxR proteins. Preferably the mutant LuxR proteins have improved regulatory activity relative to a wild-type LuxR protein. In some embodiments the mutant LuxR proteins have an extended C-terminal amino acid sequence relative to a wild type LuxR protein. The C-terminal amino acid sequence can be extended by between about 5 and about 20 amino acids (e.g., by 6 amino acids or by 15 amino acids in length relative to the wild type LuxR protein). In some embodiments the extended C-terminal amino acid sequence is VKYVSKA (amino acids 250-256 of SEQ ID NO:72) or VKYVSKAKGNSTTLD (amino acids 250-264 of SEQ ID NO:75). In other embodiments the mutant LuxR proteins have a truncated C-terminal amino acid sequence. In some of these embodiments the mutant LuxR proteins have a C-terminal amino acid sequence which is truncated by only 1, 2, 3, 4, 5, 6, 7, 8, or 9 amino acids in length relative to a wild type LuxR protein numbered according to SEQ ID NO:42. In other embodiments mutant LuxR proteins comprise an amino acid alteration at one or more of amino acid positions 8-20, wherein the amino acid positions are numbered according to SEQ ID NO:42 (e.g., an amino acid alteration at position D8; see SEQ ID NO:73).

The invention also provides isolated nucleic acid molecules which encodes mutant LuxR proteins of the invention (e.g., SEQ ID NOS:144-149).

In other embodiments the invention provides isolated nucleic acid molecules comprising a nucleotide sequence which encodes a V. fischeri LuxI protein or a V. fischeri LuxR protein, wherein the nucleotide sequence is optimized for expression in E. coli, such as those shown in SEQ ID NOS:78-97, 133, and 134.

The invention provides expression vectors. In some embodiments the expression vectors comprise isolated nucleic acid molecules encoding LuxR mutant proteins of the invention. In other embodiments expression vectors comprise a first gene operably linked to a first promoter, wherein the first inducible is induced by a LuxR-type protein/autoinducer complex; and a second gene operably linked to a second promoter, wherein the second promoter is not induced by the LuxR-type protein/autoinducer complex and wherein expression of the second gene interferes with expression of the first gene. The first gene can encode a LuxR-type protein or it can encode a protein of interest. In some embodiments these expression vectors comprise a third promoter operably linked to a third gene encoding a LuxI-type protein (e.g., LuxI) and, if the first gene encodes a protein of interest, can also comprise a fourth promoter operably linked to a fourth gene encoding a LuxR-type protein. In any of these embodiments the LuxR-type protein can be LuxR or a mutant LuxR protein of the invention, and coding sequences can be optimized for expression in E. coli.

In other embodiments the invention provides expression vectors comprising a first gene encoding a LuxI-type protein (e.g., LuxI) operably linked to a first promoter; a second gene encoding a LuxR-type protein operably linked to a second promoter; a third gene encoding a protein of interest operably linked to a third promoter which is induced by a LuxR-type protein/autoinducer complex; and a repressor gene operably linked to a fourth promoter which is inducible but which is not induced by the LuxR-type protein/autoinducer complex, wherein expression of the repressor gene interferes with expression of luxR. In any of these embodiments the LuxR-type protein can be LuxR or a mutant LuxR protein of the invention, and coding sequences can be optimized for expression in E. coli.

The invention provides isolated host cells which comprise expression vectors of the invention. In other embodiments the invention provides isolated host cells which comprise a heterologous gene selected from the group consisting of a first gene encoding a LuxI-type protein (e.g., LuxI) and a second gene encoding a LuxR-type protein (e.g., LuxR or a mutant LuxR protein of the invention, wherein the heterologous gene is stably integrated into the genome of the isolated host cell. In embodiments in which the host cell comprises a stably integrated gene encoding the LuxI-type protein, the gene encoding the LuxR-type protein can be stably integrated into the genome of the isolated host cell or it can be provided on an expression vector. Host cells of the invention can comprise an expression vector which comprises a gene of interest operably linked to an inducible promoter, wherein the inducible promoter is induced by the LuxR-type protein/autoinducer complex. In any of these embodiments, any of the genes can be optimized for expression in E. coli.

In other embodiments the invention provides isolated host cells comprising a heterologous gene encoding a LuxR-type protein; and an expression vector encoding a gene of interest operably linked to a promoter which is induced by a LuxR-type protein/autoinducer complex. The heterologous gene can be present in an expression vector or can be stably integrated into the genome of the host cell. The heterologous gene can, for example, encode LuxR or a mutant LuxR protein of the invention. The heterologous gene or the gene of interest can be optimized for expression in E. coli.

The invention provides methods of expressing a gene of interest in a host cell of the invention. The host cell is cultured under conditions which permit expression of the gene of interest. The method can include preparing inoculum of a host cell which comprises an expression vector comprising (i) a first heterologous gene of interest operably linked to a first promoter which is responsive to induction by the LuxR autoinducer complex; and (ii) an inducible second promoter driving expression of a second gene such that expression of the second gene interferes with expression of the heterologous gene, and wherein suppression of the gene of interest during the inoculum phase is attained by inducing activation of the inducible second promoter. The inoculum is used to prepare a culture of the host cell. The recombinant protein expressed by the gene of interest can be purified and, if desired, formulated into a pharmaceutical composition (e.g., a vaccine composition).

The invention provides recombinant proteins produced as described herein, as well as pharmaceutical compositions comprising the recombinant proteins (e.g., vaccine compositions).

The invention also provides methods of optimizing expression of V. fischeri luxI or luxR genes. The method comprises obtaining a nucleotide sequence encoding LuxI or LuxR; and modifying the polynucleotide sequence to optimize codon usage in E. coli.

### BRIEF DESCRIPTION OF THE DRAWINGS

FIG. 1. Lux operon fragment amplified from V. fischeri ATCC7744 genomic DNA.

FIG. 2. pGLlux506 vector.

FIG. 3. Scheme for the construction of pLAIR32 and pLAIET32 vectors.

FIGS. 4A-B. FIG. 4A, Organization of the two convergent promoters PT7 and PluxR. FIG. 4B, the induction of the T7 promoter in BL21DE3 strain which have been grown on LB agar with 1 mM IPTG repressed the luxR expression, and so the auto-induction system and consequently the expression of Gfp protein.

FIGS. 5A-B. Codon usage optimization in luxI gene. FIG. 5A, original sequence; FIG. 5B, optimized sequence.

FIGS. 6A-B. Codon usage optimization in luxR gene. FIG. 6A, original sequence; FIG. 6B, optimized sequence.

FIGS. 7A-B. pMKSal expression vector. FIG. 7A, main features of pMKSal vector; FIG. 7B, features of the multiple cloning sites.

FIG. 8. Expression of the gfp gene by the pLAI-GFP and pMKSal-GFP in the auto-induction system. pMKSal harbored the optimized sequences of luxR and luxI genes. pLAI(−) is the negative control and do not have the gfp gene.

FIG. 9. Optimization of the “lux operon fragment” (luxR, luxI, cis-acting element in between these two genes) by Error Prone PCR. Examples of clones having a fluorescence expression from gfp reporter gene with a Quorum sensing behavior.

FIGS. 10A-C. Molecular characterization of MM294.1::luxI strain. FIG. 10A, PCR product using the LuxI4Fr\LuxI4Rv primers and MM294.1 genomic DNA as template (Lane 1: negative control, lane 2: positive control pGLLux506 plasmidic DNA, lane 3: MM294.1::luxI genomic DNA. FIG. 10B, Southern Blotting using the PCR fragment described in FIG. 10A as probe, In lanes 1 and 2 PCR product as in FIG. 10A, Lane 3 pGLEM-luxI plasmidic DNA, Lane 4 MM294.1::luxI genomic DNA, Lane 5 MM294.1 genomic DNA. The DNA was digested by XmaI and AatII restriction enzymes. FIG. 10C, luxI cassette in MM294.1 genomic DNA.

FIG. 11. pMKSal-ΔluxI vector.

FIG. 12A. Different plasmid/host strain combinations to test the expression of Gfp protein. FIG. 12B, cell culture were normalised by growth in pre-culture until saturation and then diluted in fresh medium. Fluorescence of Gfp protein was measured during the cell growth.

FIG. 13. Representations of wild-type and mutated LuxR proteins.

FIG. 14 Expression of ExPEC ΔG-3526 antigen using the vector pMKSal ΔG-3526 in E. coli HK 100 host. SDS-PAGE stained with Coomassie Blue. Lanes 1-3 correspond to the total proteins, Lane 4 correspond to the total protein of HK100/pMKSal (negative control).

### DETAILED DESCRIPTION OF THE INVENTION

The present invention provides systems for expressing recombinant proteins of interest. One advantage of these systems is that they do not rely on an exogenous activation but are self-inducible. Thus, as opposed to switching on gene expression through the addition of an exogenous inducer, the invention allows for a host cell to generate an endogenous source of an inducer in a controlled fashion, such that recombinant gene expression is triggered at a desired phase of host cell culture (and at a desired host cell density). The self-inducible aspect is achieved by using elements of the quorum sensing (QS) system of bacteria, in particular of Gram negative bacteria such as Vibrio fischeri (lux bioluminescence genes), Pseudomonas aeruginosa (virulence genes), Agrobacterium tumefaciens (conjugal transfer), Serratia liquefaciens (swarming motility), and Erwinia caratovora (antibiotic production), for example. See Fuqua and Greenberg, Curr. Opinion Microbiol. 1: 183 189, 1998; and Fuqua et al., Ann. Rev. Microbiol. 50:727 751, 1996).

The term “gene” as used herein, means a coding sequence for a protein. It can but does not necessarily include elements found in and/or associated with a gene encoding that protein in nature (e.g., introns and regulatory elements). A “heterologous gene” is a gene from a different organism than the host cell in which it is contained. A “heterologous protein” is a protein produced by a heterologous gene.

Quorum Sensing Machinery

Although the invention is not limited to use of V. fischeri QS machinery, that system is described here to illustrate aspects of the invention.

The lux bioluminescent genes of V. fischeri are activated by QS via positive feedback regulation. The lux genes are transcribed by two divergent operons which are separated by a common regulatory region. The left operon contains the luxR gene which encodes the regulatory protein LuxR. The right operon contains at least 6 genes (luxICDABE). The gene luxI encodes an autoinducer synthase (LuxI) which produces the autoinducer N-(3-oxohexanoyl)-homoserine lactone (HSL; also known as AHL and as VAI-1). LuxR binds to HSL, and the complex LuxR/HSL (also referred to herein as a “LuxR-autoinducer complex”) binds upstream of the luxICDABE operon, which allows the transcription of genes involved in the synthesis of luciferase as well an exponential transcription of luxI in a positive feedback loops. LuxR also binds to the luxR promoter, which inhibits the synthesis of LuxR (March and Bentley, Curr Opin Biotechnol. 2004 15:495-502). The luxR gene is also positively controlled by cAMP/CRP (cAMP Receptor Protein) complex, which binds to the CRP box present in the common regulatory region. The QS system is also regulated by catabolic repression. The genes of the natural QS system have been expressed in E. coil and its components were used to demonstrate cell-cell communication in this host (Engebrecht, Cell 1983, 32: 773-781; Devine et al., Proc Natl Acad Sci USA. 1989 86:5688-92).

A number of bacteria with proteins homologous to LuxR and LuxI also produce HSL autoinducers similar or identical to HSL of V. fischeri (see Table 1) and which form LuxR-type protein/autoinducer complexes which can be used in the practice of the invention.

TABLE 1

| | | | | GenBank |
|---|---|---|---|---|
| Organism | HSL synthase | HSL receptor | signal molecule | Accession Nos. |
| Aeromonas | AhyI (e.g., SEQ ID | AhyR (e.g., SEQ | N-(butyryl)-L-homoserine | X89469 |
| hydrophilia | NO: 1) | ID NO: 2) | lactone (BHL), N- | |
| | | | (hexanoyl)-L-homoserine | |
| | | | lactone (HHL) | |
| Aeromonas | AsaI (e.g., SEQ ID | AsaR (e.g., SEQ | BHL, HHL | U65741 |
| salmonicida | NO: 3) | ID NO: 4) | | |
| Agrobacterium | TraI (e.g., SEQ ID | TraR (e.g., SEQ | N-(oxooctanoyl)-L- | L17024, |
| tumefaciens | NOS: 5, 6) | ID NO: 7), TraM | homoserine lactone | L22207, |
| | | (e.g., SEQ ID | (OOHL) | AF065246, |
| | | NO: 8) | | ABB59514.1 |
| Chromobacterium | CviI (e.g., SEQ ID | CviR (e.g., SEQ | HHL | AY277257 |
| violaceum | NO: 9) | ID NO: 10) | | |
| Enterobacter | EagI (e.g., SEQ ID | EagR | N-e-(oxohexanoyl)- | X74300 |
| agglomerans | NO: 11) | | homoserine lactone | |
| | | | (OHHL) | |
| Erwinia cartovora | CarI (ExpI) (e.g., | CarR (e.g., SEQ | OHHL | U17224, |
| subsp. cartotovora | SEQ ID NO: 13) | ID NO: 12) | | X74299 |
| Erwinia | ExpI (e.g., SEQ ID | ExpR (e.g., SEQ | | X96440 |
| chrysanthemi | NO: 14) | ID NO: 15) | | |
| | EchI (e.g., SEQ ID | EchR (e.g., SEQ | | U45854 |
| | NO: 16) | ID NO: 17) | | |
| Erwinia sterwartii | EsaI (e.g., SEQ ID | EsaR (e.g., SEQ | OHHL | L32183, |
| | NO: 18) | ID NO: 19) | | L32184 |
| Escherichia coli | | SdiA (e.g., SEQ | | YP_001458706 |
| | | ID NO: 20) | | |
| Nitrosomonas | | | OHHL | |
| europaea | | | | |
| Obesumbacterium | OprI | OprR | OHHL | |
| proteus | | | | |
| Pseudomonas | RhII (e.g., SEQ ID | RhlR (e.g., SEQ | N-(oxododecanyoyl)-L- | M59425, |
| aeruginosa | NO: 22); VsmI (e.g., | ID NO: 21); VsmR | homoserine lactone | SwissProt |
| | SEQ ID NO: 23) | (e.g., SEQ ID | (OdDHL) | P33993, |
| | | NO: 24) | | L08962, |
| | | | | U11811, |
| | | | | U15644 |
| Pseudomonas | PhzI (e.g., SEQ ID | PhzR (e.g., SEQ | | L32729, |
| aeureofaciens | NO: 26) | ID NO: 25) | | L33724 |
| Pseudomonas | PhzI (e.g., SEQ ID | PhzR (e.g., SEQ | HHL | L48616 |
| fluorescens | NO: 27) | ID NO: 28) | | |
| Pseudomonas | PsyI (e.g., SEQ ID | PsyR (e.g., SEQ | | AF110468 |
| syringae pv tabaci | NO: 29) | ID NO: 30) | | |
| Ralstonia | SolI (e.g., SEQ ID | SolR (e.g., SEQ | | AF021840 |
| solancearum | NO: 31) | ID NO: 32) | | |
| Rhizobium | | RhiR (e.g., SEQ | N-(3R-hydroxy-7-cis- | M98835 |
| leguminosarum | | ID NO: 33) | tetradecanoyl)-L- | |
| | | | homoserine lactone, small | |
| | | | bacteriocin | |
| Rhodobacter | CerI (e.g., SEQ ID | CerR (e.g., SEQ | | AF016298 |
| sphaeroides | NO: 34) | ID NO: 35) | | |
| Serratia | SwrI (e.g., SEQ ID | | BHL | U22823 |
| liquifaciens | NO: 37) | | | |
| Vibrio | VanI (e.g., SEQ ID | VanR (e.g., SEQ | N-(oxodecanoyl)-L- | U69677 |
| anguillarum | NO: 37) | ID NO: 38) | homoserine lactone | |
| | | | (ODHL) | |
| Vibrio fischeri | LuxI (e.g., SEQ ID | LuxR (e.g., SEQ | OHHL, HHL | M19039, |
| | NOS: 39, 40) | ID NOS: 42, 43) | N-(octanoyl)-L- | M96844, |
| | AinI e.g., SEQ ID | AinR (e.g., SEQ | homoserine lactone (OHL) | M25752 |
| | NO: 41) | ID NO: 44) | | L37404 |
| Vibrio harveyi | LuxL (e.g., SEQ ID | LuxN (e.g., SEQ | N-(hydroxybutyryl)-L- | L13940 |
| | NO: 45) | ID NO: 47) | homoserine lactone | |
| | LuxM (e.g., SEQ | | (HBHL) | |
| | ID NO: 46) | | | |
| Xenorhabdus | | | HBHL or a close homolog | |
| nematophilus | | | | |
| Yersinia | YenI (e.g., SEQ ID | YenR (e.g., SEQ | OHHL, HHL | X76082 |
| enterocolitica | NO: 49) | ID NO: 48) | | |
| Yersinia | YepI | YepR | OHLL, HHL | |
| pseudotuberculosis | | | | |
| Yersinia ruckeri | YukI | YukR | | |

The signal molecules listed in Table 1 have identical homoserine lactone moieties but can differ in the length and structure of their acyl groups. LuxI and corresponding enzymes from other species catalyze the ligation of S-adenosylmethionine (SAM) and a fatty acyl chain derived from acyl-acyl carrier protein (ACP) conjugates.

“LuxR-type proteins” according to the invention typically are composed of two modules, an amino-terminal domain (residues 1 to 160 of LuxR, numbered according to SEQ ID NO:42) with an HSL-binding region (residues 79-127 of LuxR, numbered according to SEQ ID NO:42) and a carboxy-terminal transcription regulation domain (residues 160-250 of LuxR, numbered according to SEQ ID NO:42), which includes a helix-turn-helix DNA-binding motif (residues 200-224 of LuxR, numbered according to SEQ ID NO:42). The carboxy-terminal one-third of these proteins is homologous to DNA binding domains of the LuxR superfamily of transcriptional regulators.

“LuxI-type proteins” according to the invention are proteins which produce an autoinducer (such as those listed in Table 1) which binds to a LuxR-type protein to form a LuxR-type protein/autoinducer complex. A “LuxR-type protein/autoinducer complex” activates gene expression at a certain cell density which corresponds to a threshold concentration of autoinducer. A general mechanism of activation for this superfamily of proteins has been proposed; see U.S. Pat. No. 7,202,085.

LuxR binds as a homomultimer to the LuxR binding site, which has a dyad symmetry, and a region required for multimerization resides within amino acids 116 and 161 of the amino-terminal portion of the protein, numbered according to SEQ ID NO:42.

The LuxR binding site, or lux box (5′-ACCTGTAGGATCGTACAGGT-3′ SEQ ID NO:50), is a 20-nucleotide inverted repeat centered 44 nucleotides upstream of the transcription start site of the luminescence operon (Devine et al., Proc. Natl. Acad. Sci. USA 86:5688 5692, 1989; Gray et al., J. Bacteriol. 176:3076 3080, 1994). Similarly, 18-bp tra boxes are found upstream of at least three TraR-regulated promoters and are required for transcriptional activation by TraR (Fuqua and Winans, J. Bacteriol. 178:435 440, 1996). Similar sequences found in LasR-regulated promoters invariably overlap putative −35 elements of σ⁷⁰-type promoters by one nucleotide. The lax and las boxes are sufficiently similar that LuxR can activate transcription from the lasB promoter in the presence of HSL, and conversely, LasR can activate transcription of the luminescence operon in the presence of PAI—I (Gray et al., J. Bacteriol. 176:3076 3080, 1994). A number of lux box-like sequences (also referred to herein as “HSL response elements”) have been compared (Table 2). The consensus lux box-like sequence is 5′-RNSTGYA-GATN-TRCASRT-3′ (SEQ ID NO:51). Synthetic HSL response elements may be produced by varying one or more nucleotides of a native lux box-like sequence. For example, as discussed U.S. Pat. No. 7,202,085, when TraR is expressed in carrot cells, a promoter that includes the traA box shows a higher than expected level of basal activity. This basal activity can be significantly reduced without eliminating HSL responsiveness by replacing the traA box with a variant box in which a small number of base pairs of the traA box are altered.

Synthetic HSL-responsive promoters may be produced by replacing an HSL response element from one promoter with an HSL-response element from another promoter, or by adding a native or synthetic HSL-response element to a promoter that lacks a functional HSL response element, such as a minimal promoter. In addition, two or more HSL response elements may be present in a single promoter to render the promoter responsive to more than one HSL. A promoter that comprises one or more HSL-response elements is referred to herein as an “HSL-responsive promoter.”

TABLE 2

| gene | lux box-like sequence | SEQ ID NO: |
|---|---|---|
| luxI | ACCTGTAGGATCGTACAGGT | 52 |
| luxD | GAATGGATCATTTTGCAGGT | 53 |
| lasB | ACCTGCCAGTTCTGGCAGGT | 54 |
| esaR | ACCTGCACTATAGTACAGGC | 55 |
| cepI | CCCTGTAAGAGTTACCAGTT | 56 |
| solI | CCCTGTCAATCCTGACAGTT | 57 |
| rhlI | CCCTACCAGATCTGGCAGGT | 58 |
| traI1 | ACGTGCA-GATC-TGCACAT | 59 |
| traI2 | AAGTGCA-GATT-TGCACAT | 60 |
| traA | ATGTGCA-GATC-TGCACAT | 61 |
| traA1 | gTGTGCA-GATC-TGCACAc | 62 |
| traA2 | AgGTGCA-GATC-TGCACcT | 63 |
| traA3 | ATtTGCA-GATC-TGCAaAT | 64 |
| traA4 | ATGaGCA-GATC-TGCtCAT | 65 |
| traA5 | ATGTcCA-GATC-TGgACAT | 66 |
| traA6 | ATGTGaA-GATC-TtCACAT | 67 |
| traA7 | ATGTGCg-GATC-cGCACAT | 68 |
| traA8 | ATGTGCA-aATt-TGCACAT | 69 |
| traA9 | ATGTGCA-GtaC-TGCACAT | 70 |
| traA-1 | ATGTGCA-GA-C-TGCACAT | 71 |

Other promoters regulated by TraR and LasR lack these sites. For example, the lasI gene does not have a recognizable las box upstream of its promoter (Passador et al., Science 260:1127 1130, 1993) and yet is strongly inducible by LasR. Similarly, the traM gene of A. tumefaciens appears to have two half-sites upstream of its promoter rather than an orthodox tra box (Fuqua et al., J. Bacteriol. 177:1367 1373, 1995; Hwang et al., J. Bacteriol. 177:449 458, 1995) and is mildly inducible by TraR. The TraR protein also activates expression of the traR gene at a promoter that has no apparent similarity to any tra box motif In the case of TraR promoters that have a strong similarity to the consensus tra box motifs are activated to high level expression by 3-oxooctanoyl-homoserine lactone (AAI), and more degenerate motifs are associated with lower levels of induction.

Quorum-sensing promoters may be altered to make them responsive to a different HSL autoinducer by “operator swapping,” that is, by replacing lux box-like sequence(s) from the promoter with a lux box-like sequence from a different promoter. For example, a lux box sequence in one promoter may be replaced by a tra or las box sequence. HSL responsiveness can also be modified by “domain swapping,” that is, by replacing an HSL-binding region of one LuxR-like protein with the HSL-binding region of another LuxR-like protein such that the DNA-binding specificity of the resulting chimeric protein is unchanged. For example, replacement of the HSL-binding region of LuxR with the HSL-binding region of TraR would cause the resulting chimeric protein to bind the lux box sequence and modulate transcriptional activity in response to binding of the autoinducer HSL. In addition, the activation domain of a LuxR-like protein can be replaced by another activation domain that is a well known activator of gene expression in a given host cell, such as GAL4, VP16, or other well known activator domains.

New members of the LuxR-LuxI family have been sought by screening bacteria for the release of autoinducers using an Escherichia coli strain containing a cloned lux regulon but lacking luxI (and therefore not synthesizing HSL). Similar experiments have been performed with Agrobacterium tumefaciens TraR regulator to screen plant pathogenic soil bacteria. These studies have demonstrated that LuxR and TraR are activated by a subset of known autoinducers. It has also been demonstrated that LuxR-like proteins such as LuxR and Pseudomonas aeruginosa LasR activate lux gene expression after binding derivatives of the cognate autoinducers with alterations in acyl chain length or in the carbonyl groups, for example (Eberhard et al., Arch. Microbiol. 146:35 40, 1986; Kuo et al., J. Bacteriol. 176:7558 7565, 1994; Kuo et al., J. Bacteriol. 178:971976, 1996; Pearson et al., Proc. Natl. Acad. Sci. USA 91:197 201, 1994; Fuqua and Winans, J. Bacteriol. 176:2796 2806, 1994).

EsaR, ExpR, and YenR are reported to be repressors of their target genes rather than activators, and their respective autoinducers increase expression of the repressed genes, which can be useful to derepress a gene at high cell density.
Mutated LuxR Proteins and Nucleic Acid Molecules Encoding Mutant LuxR Proteins

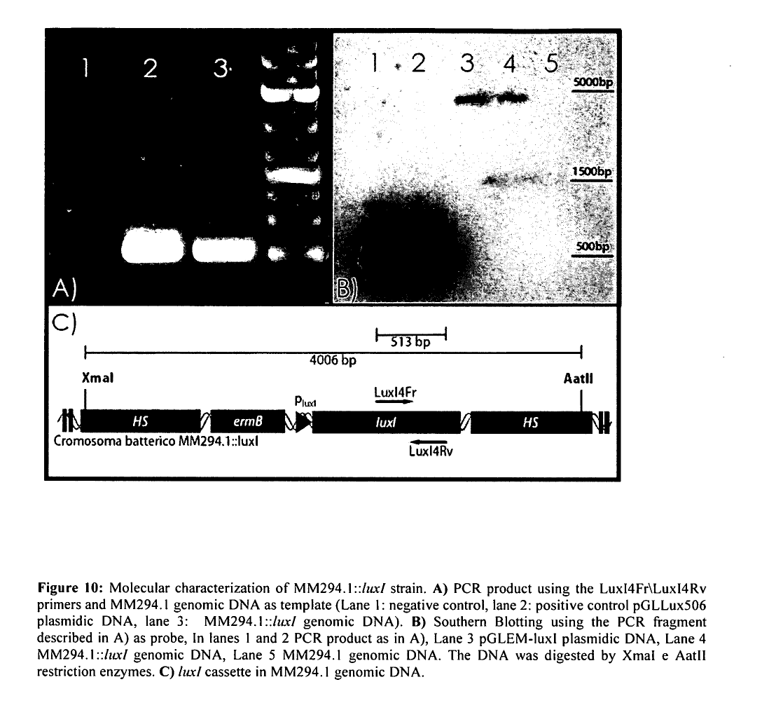

The invention provides mutated LuxR proteins and isolated nucleic acid molecules encoding the mutated LuxR proteins. Mutated LuxR proteins according to the invention can exhibit improved regulatory activity relative to a wild type LuxR protein and can therefore be used to optimize control of expression of a gene of interest. A mutated LuxR protein has “improved regulatory activity” if it has one or more of the following effects: (1) a lower basal level of induction compared with that of a wild-type LuxR; (2) a stronger level of induction compared with that of a wild-type LuxR; and (3) delayed induction compared to that of a wild-type LuxR. Examples of mutant LuxR proteins with improved regulatory activity are described in Example 7 and in FIG. 10. Use of nucleic acid molecules comprising mutated sequences result in an altered basal level of expression of a gene of interest and/or they increase the strength of autoinduction strength (i.e., a more rapid or a higher expression level after autoinduction is triggered).

In some embodiments the mutated LuxR proteins have a lengthened C terminus. In some embodiments the C terminus is lengthened by between 1 and 20 amino acids (e.g., between 5 and 20 amino acids; between 5 and 10 amino acids; between 5 and 15 amino acids; or an addition of 1, 2, 3, 4, 5, 6, 7, 8, 9, 10, 11, 12, 13, 14, 15, 16, 17, 18, 19, or 20 amino acids). In some embodiments the C terminus of the mutant LuxR is lengthened by 6 or by 15 amino acids; examples of such mutant LuxR proteins are shown in SEQ ID NO:72 and SEQ ID NO:75.

LuxR 1M37-2M27-2M28
(SEQ ID NO: 72)
MKDINADDTYRIINKIKACRSNNDINQCLSDMTKMVHCEYYLLAIIYPHSMVKSDISILDNYPKKWRQYYDDANL
IKYDPIVDYSNSNHSPINWNIFENNAVNKKSPNVIKEAKSSGLITGFSFPIHTANNGFGMLSFAHSEKDNYIDSL
FLHACMNIPLIVPSLVDNYRKINIANNKSNNDLTKREKECLAWACEGKSSWDISKILGCSKRTVTFHLTNAQMKL
NTTNRCQSISKAILTGAIDCPYFKVKYVSKA
LuxR 2M15
(SEQ ID NO: 75)
MKDINADDTYRIINKIKACRSNNDINQCLSDMTKMVHCEYYLLAIIYPHSMVKSDISILDNYPKKWRQYYDDANL
IKYDPIVDYSNSNHSPINWNIFENNAVNKKSPNVIKEAKSSGLITGFSFPIHTANNGFGMLSFAHSEKDNYIDSL
FLHACMNIPLIVPSLVDNYRKINIANNKSNNDLTKREKECLAWACEGKSSWDISKILGCSKRTVTFHLTNAQMKL
NTTNRCQSISKAILTGAIDCPYFKVKYVSKAKGNSTTLD

In some cases a mutant LuxR protein has a C terminal truncation of 1-10 contiguous amino acids at the C terminus and an improved regulatory activity (e.g., 1, 2, 3, 4, 5, 6, 7, 8, 9, or 10). The deleted amino acids preferably are contiguous. In other cases, only 0.1, 2, 3, 4, 5, 6, 7, 8, or 9 amino acids at the C terminus are removed from LuxR; an example of this type of mutant LuxR protein is shown in SEQ ID NO:74.

LuxR 1M27
(SEQ ID NO: 74)
MKDINADDTYRIINKIKACRSNNDINQCLSDMTKMVHCEYYLLAIIYPHSMVKSDISILDNYPKKWRQYYDDANL
IKYDPIVDYSNSNHSPINWNIFENNAVNKKSPNVIKEAKSSGLITGFSFPIHTANNGFGMLSFAHSEKDNYIDSL
FLHACMNIPLIVPSLVDNYRKINIANNKSNNDLTKREKECLAWACEGKSSWDISKILGCSKRTVTFHLTNAQMKL
NTTNRCQSISKAILTGAIDCPYF--

In other cases altered amino acids are present in the autoregulatory region of mutant LuxR between amino acid positions 8 and 20, numbered according to the LuxR wild-type amino acid sequence shown in SEQ ID NO:42. In some embodiments amino acid position 8 has an altered amino acid; an example of this type of mutant LuxR protein is shown in SEQ ID NO:73.

LuxR 1M16
(SEQ ID NO: 73)
MKDINADNTYRIINKIKACRSNNDINQCLSDMTKMVHCEYYLLAIIYPHSMVKSDISILDNYPKKWRQYYDDANL
IKYDPIVDYSNSNHSPINWNIFENNAVNKKSPNVIKEAKSSGLITGFSFPIHTANNGFGMLSFAHSEKDNYIDSL
FLHACMNIPLIVPSLVDNYRKINIANNKSNNDLTKREKECLAWACEGKSSWDISKILGCSKRTVTFHLTNAQMKL
NTTNRCQSISKAILTGAIDCPYFKS

The invention provides isolated nucleic acid molecule comprising coding sequences for the mutant LuxR proteins described above. Examples of such coding sequences are shown in SEQ ID NOS:144-149 (reverse complements of SEQ ID NOS:138-143. As explained in the specific Examples, below, mutant coding sequences can be obtained using Error prone PCR Random mutagenesis as described in Cadwell & Joyce, PCR Methods Appl. 1992 August; 2(1):28-33. See Example 7 and Table 12.

Codon-Optimized Genes

In some embodiments the nucleic acid molecules comprise altered coding sequences which do not affect the amino acid sequence of LuxR but which have an effect on expression kinetics. Many problems in expressing a heterologous gene in a foreign host strain are the result of the difference between the codon usage between the host strain (e.g., E. coli) and the strain from which the heterologous protein is native. Rare codons can especially be a problem. Amino acids are encoded by more than one codon, and each organism has a preference in the use of codons, also known as codon usage bias. The tRNA population reveal the codon bias in a determined cell (Dong (1996) J Mol Biol 260:649-663). The overexpression of an heterologous protein, in which some tRNAs that may be rare or lacking in the expression host, can impede its expression. This can eventually cause for example translational stalling, premature translation termination, translation frameshift, and amino acid misincorporation (Kurland and Gallant (1996) Curr Opin Biotechnol 7:489-493).

Analysis of the nucleotide sequence of luxR shows that the translation of luxR and luxI mRNA requires the use of rare tRNA in E. coli. The invention therefore provides nucleic acid molecules comprising a polynucleotide sequence of luxR or luxI from V. fischeri in which one or more codons of the coding sequence are optimized for expression of LuxI and/or LuxR in a host cell, preferably E. coli. Preferably the entire polynucleotide sequence is codon-optimized (i.e., as many codons as possible are altered for optimized expression). Benefits associated with optimized sequence include the fact that full expression of regulative elements does not limit the regulation and the expression of target gene from the luxI promoter. Modification of restriction sites provides the option of having unique restriction sites in plasmids. Expression vectors containing codon-optimized sequences are very efficient for large-scale production with improved efficiency for expression of a gene of interest.

A wild-type LuxR-encoding sequence is shown in SEQ ID NO:77. Examples of codon-optimized LuxR-encoding sequences are provided as SEQ ID NOS:78-88 and 133. A wild-type LuxI-encoding sequence is shown in SEQ ID NO:76. Examples of codon-optimized LuxI-encoding sequences are provided as SEQ ID NOS:89-97 and 134.

The invention also provides a method for optimizing expression of LuxR or LuxI in a host cell (e.g., E. coli). The method comprises (i) obtaining a polynucleotide sequence of luxR or luxI; and (ii) modifying the polynucleotide sequence to optimize for codon usage in the host cell. Optimization can be in particular obtained by modifications of the sequence of the luxR and/or luxI genes to enhance compatibility with the codon usage of a particular host cell. Codon-optimization methods can also be used to obtain codon-optimized sequences which encode LuxI-like and LuxR-like proteins (e.g., as listed in Table 1).

Expression Vectors

Elements of QS machinery such as those described above can be used to construct expression vectors (e.g., plasmids) for transformation of a host cell. These expression vectors can be used in methods of the invention, in particular methods which rely on transcriptional interference. “Transcriptional interference” is the perturbation of one transcription unit by another. Transcriptional interference can have an influence, generally suppressive, of one active transcriptional unit on another transcriptional unit linked in cis. The studies of Eszterhas et al. (2002, Mol. Cell. Biol. 22, 469-479) suggested that two closely linked transcription units will always interfere with each other. It was shown that two promoters which are faced (also called convergent promoters) showed the most significant interference compare to promoters which are in tandem (in same direction) or divergent (opposite direction). Transcriptional interference has been studied in E. coli and in eukaryotic cells (Padidam and Cao, 2001; Eszterhas et al., 2002; Prescott and Proudfoot, 2002). See Shearwin et al., TRENDS in Genetics 21, 339-45. 2005.

In the description which follows, LuxI and LuxR are used as examples; however, the invention explicitly encompasses similar embodiments in which other QS machinery is used (e.g., LuxR- and LuxI-type proteins as defined above, including those listed in Table 1).

It is known that LuxR is necessary for the regulation of gene expression under the P_{luxI} promoter (Shadel et al., 1992). In one embodiment of the invention, the promoter used for the transcription interference is an inducible promoter such as P_{lac}, P_{bad}, P_{tac}, P_{tcr}, Pₜᵣₚ, and Pₘₑₜ. Other inducible promoters include ADH2, GAL-1-10, GAL 7, PHO5, T7, T5, and metallothionine promoters. Other examples of inducible promoters are listed in Table 3. These lists are not exhaustive. The promoter which interferes can be convergently, in tandem, or divergently oriented with respect to the promoter to be repressed. When the promoter to be repressed is the promoter of luxR gene; the promoter which interferes preferably is convergently oriented. It is suitably located upstream to luxR gene, preferably upstream. When the promoter to be repressed is the LuxR/autoinducer promoter, the promoter which interferes preferably is convergently oriented. It is suitably located upstream to gene of interest, preferably upstream.

TABLE 3

| Promoter | Regulator | Induction |
|---|---|---|
| Inducible | | |
| lac | LacI, LacIts | isopropyl-β-D- |
| | | thiogalactoside |
| | | (IPTG), |
| | | temperature |
| trc and tac | LacI, LacIts | IPTG, temperature |
| λPₗ | λcIts 857 | temperature |
| T7 | LacI, LacIts | IPTG, temperature |
| P_{BAD} | AraC | L-arabinose |
| cspA (Mujacic et al. (1999) | | temperature |
| Gene. 238(2): 325-332) | | |
| rhaPBAD | RhaR, RhaS | L-ramnose |
| tetA | TetR | Tetracyclin |
| Pm | XylS | m-toluic acid |
| lpp (Nakamura, K., and M. Inouye. | | IPTG, lactose |
| 1982 EMBO J. | | |
| 1: 771-775) | | |
| recA | LexA | Nalidixic acid |
| Psyn (Giacobini et al. 1994. | LacI, LacIq | IPTG |
| Gene 144: 17-24) | | |
| pL-9G-50 (Oppenheim et | | Temperature |
| al., WO 96/03521) | | (<20° C.) |
| T3-lac operator | LacIq, | IPTG |
| T5-lac operator | LacIq, LacI | IPTG |
| T4 gene 32 (Gorski et al.. | | Infection T4 |
| 1985 Cell 43: 461-469) | | |
| nprM-lac operator | LacIq | IPTG |
| (Yamada et al. 1991 Gene | | |
| 99: 109-114) | | |
| Protein A | | |
| Regulated by nutrients | | |
| phoA | PhoB, PhoR | Phosphate |
| | | depletion |
| ugpBAECQ (Su et al., 1991 | | Glucose and |
| Mol. Gen. Genet. 230, 28-32) | | phosphate |
| | | depletion |
| proU (Herbst et al., 1994 | | Osmolarity (NaCl) |
| Gene 151: 137-142) | | |
| vglnAp2 (Schroeckh et al. | | Nitrogen depletion |
| 1992, DE 4102594) | | |
| trp | | Tryptophan |
| | | depletion, IAA |
| cst-1 | | Glucose depletion |
| mglBAEC (Death and | MglD | Addition of |
| Ferenci 1994. J. Bacteriol. | | glucose |
| 176, 5101-5107) | | |
| araB | | Glucose depletion |
| | | and arabinose |
| | | addition |
| Starvation promoters | | |
| (Matin. 1994, Ann. N. Y. | | |
| Acad. Sci. 721: 277-291) | | |
| Regulated by oxygen | | |
| vhb (Dikshit et al. 1990. | | Microanaerobic, |
| Acids Res. 18, 4149-4155) | | cAMP-CAP |
| pfl (Oxer et al. 1991 Nucleic | | Anaerobic |
| Acids Res. 19, 2889-2892) | | |
| nirB | Fnr (FNR, | Anaerobic |
| | NARL) | |
| nar | | Anaerobic |
| Regulated by pH | | |
| cadA | CadR | Acid pH |

For example, the inhibition of the auto-induction system at high cell density can be obtained by inhibiting the expression of luxR. For this purpose, an inducible promoter like the T7 promoter can inserted upstream of the luxR gene and in a convergent orientation to the promoter of luxR gene in the auto-inducible expression vector. This vector can be introduced for example in the E. coli BL21DE3 strain where the T7 promoter is inducible by the addition of IPTG in the medium, and the repression of luxR by transcriptional interference can be observed.

A wide variety of expression vectors are commercially available and can be used to produce expression vectors of the invention. Alternatively, expression vectors can be constructed using recombinant DNA methods long known in the art. These vectors include, but are not limited to plasmids, cosmids, Bac, Pac, bacteriophage, transposable elements and transient expression system. The vector can be a low, medium or a high copy number plasmid Preferred expression vectors include, but are not limited to, pSM214G, pKMSal, pLAIET32, pLAIR32, pLAIET42, pLAIR42, pGlowlux506, pGLEM, pGlow, pKMluxI-, and pET21.

In some embodiments, an expression vector comprises (1) a first gene operably linked to a first inducible promoter which is inducible by a LuxR/autoinducer complex; and (2) a second gene operably linked to a second inducible promoter, wherein the second inducible promoter is not induced by the LuxR/autoinducer complex. In such expression vectors, expression of the second gene interferes with expression of the first gene by means of transcriptional interference. In some embodiments the first gene encodes the LuxR. In other embodiments the first gene encodes a protein of interest. When luxR is present in an expression vector, the inducible second promoter may be oriented such that activation of the promoter interferes with expression of luxR.

In yet another embodiment the vector further comprises luxI. Preferably luxI is operably linked to a third promoter which responds to induction by the LuxR autoinducer complex. In some embodiments an expression vector encodes both LuxR and the protein of interest. In other embodiments, V. fischiae luxI and luxR genes are present on single or separate expression vectors, while the gene of interest, operably linked to the luxI promoter, is present in an expression vector.

Proteins of Interest

Any protein of interest can be expressed using methods of the invention. The protein of interest may be any eukaryotic and prokaryotic polypeptide, such as for example proteins from mammals, plants, yeast, fungi, bacteria, archeobacteria, protozoa, algae, viruses, and phage. The protein of interest may be a prion. The protein of interest can be natural or synthetic such as for example the N19 synthetic protein (U.S. Pat. No. 6,855,321, Baraldo et al., 2004 Infect Immun. 72:4884-7). Examples of proteins which can be recombinantly produced using the invention include secretory proteins, periplasmic proteins, transmembrane proteins, cytoplasmic proteins and proteins which localize to specific organelles within the host cell.

Typically the protein of interest is an antigen, which can be used in vaccines, to stimulate immune responses. Antigens include antigens from a Gram positive bacterium (e.g., Streptococcus pyogenes, Streptococcus agalactiae, Streptococcus pneumoniae, Streptococcus equi, Staphylococcus aureus, Clostridium difficile, Clostridium tetani, Corynebacterium diphteriae, Listeria). Preferred antigens are disclosed, for example, in WO02/34771, WO03/093306, WO04/018646, WO04/041157, WO05/028618, WO05/032582, WO06/042027, WO06/069200, WO06/078318, WO02/094868, Nencioni L, 1991, Adv Exp Med. Biol. 303:119-27, WO1985/003508, and WO2007/026247 and include those listed below, as well as combinations and/or fragments thereof.

Other proteins of interest are antigens of Gram negative bacteria such as Neisseria meningitides serogroup A, B, C, W135 and Y, Neisseria gonorrhoeae, Vibrio cholerae, Haemophilus influenzae, non typeable Haemophilus, Yersinia pestis, Bordetella pertussis, enteric and Extra intestinal pathogenic strains of Escherichia coli, Moraxella catarrhalis, Helicobacter pylori, Shigella, Salmonella, Klebsiella pneumoniae, Legionella pneumophila, Pseudomonas aeruginosa, Borrelia. Coding sequences for these and other antigens of interest are disclosed, for example, in WO99/24578, WO99/36544, WO99/57280, WO00/22430, Pizza et al. (2000) Science 287:1816-1820 and WO96/29412, WO99/24578, WO99/36544, WO99/57280, WO1992/019265, WO2005/111066, WO2007/049155, WO/1989/001976, WO/1990/04641, WO2006/089264, WO2006/091517, WO2009/104092, WO2004/113371, WO2003/074553, WO2005/097823, WO2001/066572 and WO2008020330. Other antigens of interest are antigens of Chlamydia trachomatis, Chlamydia penumoniae, Plasmodium, Plasmodium falciparum, Candida albicans, Mycobacterium tuberculosis, hepatitis A virus, hepatitis B virus, hepatitis C virus, SARS-Corona Virus, Flavivirus and HIV. Coding sequences for these and other antigens of interest are disclosed, for example, in WO95/28487, WO00/37494, WO03/068811, WO03/049762, WO2005/002619, WO2006/138004, WO2007/110700, WO02/02606, WO2005/084306, W01992/019758, WO1996/004301, WO2007/041432, and WO1995/033053.

Streptococcus agalactiae (Group B Streptococcus): Group B Streptococcus antigens include a protein or saccharide antigen identified in WO 02/34771, WO 03/093306, WO 04/041157, or WO 2005/002619 (including proteins GBS 67 (SAG1408), GBS 80 (SAG0645), GBS104 (SAG0649), and GBS 322 (SAG0032), and including saccharide antigens derived from serotypes Ia, Ib, Ia/c, II, III, IV, V, VI, VII and VIII).

Streptococcus pneumoniae: Streptococcus pneumoniae antigens may include a saccharide (including a polysaccharide or an oligosaccharide) and/or protein from Streptococcus pneumoniae. Saccharide antigens may be selected from serotypes 1, 2, 3, 4, 5, 6B, 7F, 8, 9N, 9V, 10A, 11A, 12F, 14, 15B, 17F, 18C, 19A, 19F, 20, 22F, 23F, and 33F. Protein antigens may be selected from a protein identified in WO 98/18931, WO 98/18930, U.S. Pat. No. 6,699,703, U.S. Pat. No. 6,800,744, WO 97/43303, and WO 97/37026. Streptococcus pneumoniae proteins may be selected from the Poly Histidine Triad family (PhtX), the Choline Binding Protein family (CbpX), CbpX truncates, LytX family, LytX truncates, CbpX truncate-LytX truncate chimeric proteins, pneumolysin (Ply), PspA, PsaA, Sp128, Sp101, Sp130, Sp125 or Sp133.

Streptococcus pyogenes (Group A Streptococcus): Group A Streptococcus antigens may include a protein identified in WO 02/34771 or WO 2005/032582 (including, but not limited to, GAS39 (spy0266; gi-15674446), GAS40 (spy0269; gi-15674449), GAS42 (spy0287; gi-15674461), GAS45 (M5005_spy0249; gi-71910063), GAS57 (spy0416; gi-15674549), GAS58 (spy0430; gi-15674556), GAS84 (spy1274; gi-15675229), GAS95 (spt1733; gi-15675582), GAS117 (spy0448; gi-15674571), GAS130 (spy0591; gi-15674677), GAS137 (spy0652; gi-15674720), GAS159 (spy1105; gi-15675088), GAS193 (spy2025; gi-15675802), GAS202 (spy1309; gi-15675258), GAS217 (spy0925; gi-15674945), GAS236 (spy1126; gi-15675106), GAS253 (spy1524; gi-15675423), GAS277 (spy1939; gi-15675742), GAS294 (spy1173; gi-15675145), GAS309 (spy0124; gi-15674341), GAS366 (spy1525; gi-15675424), GAS372 (spy1625; gi-15675501), GAS384 (spy1874; gi-15675693), GAS389 (spy1981; gi-15675772), GAS504 (spy1751; gi-15675600), GAS509 (spy1618; gi-15675496), GAS290 (spy1959; gi-15675757), GAS511 (spy1743; gi-15675592), GAS527 (spy1204; gi-15675169), GAS529 (spy1280; gi-15675233), and GAS533 (spy1877; gi-15675696)), fusions of fragments of GAS M proteins (including those described in WO 02/094851, and Dale, Vaccine (1999) 17:193-200, and Dale, Vaccine 14(10): 944-948), fibronectin binding protein (Sfb1), Streptococcal heme-associated protein (Shp), and Streptolysin S (SagA). Further GAS antigens include GAS68 (Spy0163; gi13621456), GAS84 (Spy1274; gi13622398), GAS88 (Spy1361; gi13622470), GAS89 (Spy1390; gi13622493), GAS98 (Spy1882; gi13622916), GAS99 (Spy1979; gi13622993), GAS102 (Spy2016, gi13623025), GAS146 (Spy0763; gi13621942), GAS195 (Spy2043; gi13623043), GAS561 (Spy1134; gi13622269), GAS179 (Spy1718, gi13622773) and GAS681 (spy1152; gi1362228).

Staphylococcus aureus: Staphylococcus aureus antigens include S. aureus type 5 and capsular polysaccharides optionally conjugated to nontoxic recombinant Pseudomonas aeruginosa exotoxin A, such as StaphVAX™, or antigens derived from surface proteins, invasins (leukocidin, kinases, hyaluronidase), surface factors that inhibit phagocytic engulfment (capsule, Protein A), carotenoids, catalase production, Protein A, coagulase, clotting factor, and/or membrane-damaging toxins (optionally detoxified) that lyse eukaryotic cell membranes (hemolysins, leukotoxin, leukocidin).

In one embodiment the antigen of interest is a 3526 antigen from ExPEC as described in WO2009/104092. Optionally, the antigen of interest is the Δ3526 antigen from ExPEC as described in WO2009/104092, having the sequence as shown in SEQ ID NO 152.

In some embodiments, the invention provides proteins which are homologs, orthologs, allelic variants and/or mutants of the proteins of interest described above. These proteins comprise amino acid sequences that have sequence identity to the proteins of interest described above. Depending on the particular sequence, the degree of sequence identity is preferably 50% or more (e.g. 60%, 70%, 80%, 90%, 91%, 92%, 93%, 94%, 95%, 96%, 97%, 98%, 99%). Identity between proteins is preferably determined by Smith-Waterman homology search algorithm as implemented in the MPSRCH program (Oxford Molecular), using an affine gap search with parameters gap open penalty=12 and gap extension penalty=1. The invention includes also fragments of those proteins of interest. Preferred amino acid fragments include at least n consecutive amino acids, wherein n is 7 or more (e.g. 8, 10, 12, 14, 16, 20, 25, 30, 35, 40, 45, 50, 60, 70, 80, 90, 100, 150, 200, 250, or more).

Host Cells

The invention provides isolated host cells which comprise one or more expression vectors of the invention and which can be used to produce a protein of interest. “Isolated host cells” according to the invention are cells which have been removed from an organism and/or are maintained in vitro in substantially pure cultures.

A wide variety of cell types can be used as host cells of the invention, including both prokaryotic and eukaryotic cells. Host cells include, without limitation, bacterial cells, fungal cells, yeast cells, insect cells, and mammalian cells. Methods for introduction of heterologous polynucleotides into host cells are known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Useful bacterial host cells include Gram negative bacteria, such as Escherichia coli [Shimatake et al. (1981) Nature 292:128; Amann et al. (1985) Gene 40:183; Studier et al. (1986) J Mol. Biol. 189:113; EP-A-0 036 776, EP-A-0 136 829 and EP-A-0 136 907], Pseudomonas fluorescens, Pseudomonas haloplanctis, Pseudomonas putida AC10, Pseudomonas pseudoflava, Bartonella henselae, Pseudomonas syringae, Caulobacter crescentus, Zymomonas mobilis, Rhizobium meliloti, Myxococcus xanthus and Gram positive bacteria such as Bacillus subtilis [Palva et al. (1982) Proc. Natl. Acad. Sci. USA 79:5582; EP-A-0 036 259 and EP-A-0 063 953; WO 84/04541], Streptococcus cremoris [Powell et al. (1988) Appl. Environ. Microbiol. 54:655]; Streptococcus lividans [Powell et al. (1988) Appl. Environ. Microbiol. 54:655], and Streptomyces lividans [U.S. Pat. No. 4,745,056].

Useful fungal host cells include Aspergillis oryzae, Aspergillis niger, Trichoderma reesei, Aspergillus nidulans, Fusarium graminearum.

Useful slime mold host cells include Dictyostelium [Arya, et al. (2008) FASEB J. 22:4055.

Useful yeast host cells include Candida albicans [Kurtz, et al. (1986) Mol. Cell. Biol. 6:142], Candida maltosa [Kunze, et al. (1985) J. Basic Microbiol. 25:141]. Hansenula polymorpha [Gleeson, et al. (1986) J. Gen. Microbiol. 132:3459; Roggenkamp et al. (1986) Mol. Gen. Genet. 202:302], Kluyveromyces fragilis [Das, et al. (1984) J. Bacteriol. 158:1165], Kluyveromyces lactis [De Louvencourt et al. (1983) J. Bacteriol. 154:737; Van den Berg et al. (1990) Bio/Technology 8:135], Pichia guillerimondii [Kunze et al. (1985) J. Basic Microbiol. 25:141], Pichia pastoris [Cregg, et al. (1985) Mol. Cell. Biol. 5:3376; U.S. Pat. Nos. 4,837,148 and 4,929,555], Saccharomyces, Saccaromices cerevisiae [Hinnen et al. (1978) Proc. Natl. Acad. Sci. USA 75:1929; Ito et al. (1983) J. Bacteriol. 153:163], Schizosaccharomyces pombe [Beach and Nurse (1981) Nature 300:706], and Yarrowia lipolytica [Davidow, et al. (1985) Curr. Genet. 10:380471 Gaillardin, et al. (1985) Cum Genet. 10:49].

Methods of introducing exogenous DNA into yeast hosts are well-known in the art, and usually include either the transformation of spheroplasts or of intact yeast cells treated with alkali cations. Transformation procedures usually vary with the yeast species to be transformed. See eg. [Kurtz et al. (1986) Mol. Cell. Biol. 6:142; Kunze et al. (1985) J. Basic Microbiol. 25:141; Candida]; [Gleeson et al. (1986) J. Gen. Microbiol. 132:3459; Roggenkamp et al. (1986) Mol. Gen. Genet. 202:302; Hansenula]; [Das et al. (1984) J. Bacteriol. 158:1165; De Louvencourt et al. (1983) J. Bacteriol. 154:1165; Van den Berg et al. (1990) Bio/Technology 8:135; Kluyveromyces]; [Cregg et al. (1985) Mol. Cell. Biol. 5:3376; Kunze et al. (1985) J. Basic Microbiol. 25:141; U.S. Pat. Nos. 4,837,148 and 4,929,555; Pichia]; [Hinnen et al. (1978) Proc. Natl. Acad. Sci. USA 75; 1929; Ito et al. (1983) J. Bacteriol. 153:163 Saccharomyces]; [Beach and Nurse (1981) Nature 300:706; Schizosaccharomyces]; [Davidow et al. (1985) Curr. Genet. 10:39; Gaillardin et al. (1985) Curr. Genet. 10:49; Yarrowia].

Useful mammalian host cells include Chinese hamster ovary (CHO) cells, HeLa cells, baby hamster kidney (BHK) cells, monkey kidney cells (COS), human hepatocellular carcinoma cells (eg. Hep G2).

Useful insect host include infection with AcNPV and BmNPV in Sf9 cell line of Spodoptera fugiperda or Kc of Drosophila melanogaster.

The substrates for HSL biosynthesis by luxI are available in both prokaryotic and eukaryotic cells. In the description which follows, LuxI and LuxR are used as examples; however, the invention explicitly encompasses similar embodiments of host cells in which other QS machinery is used (e.g., LuxR- and LuxI-type proteins as defined above, including those listed in Table 1).

In one aspect of the invention, the host cell is transformed such that it encodes a functional luxI gene, a functional luxR gene and a functional luxI promoter operably linked to a gene of interest. Transformation can be carried out using any method known in the art, such as those discussed above. During host cell culture, the luxI gene is expressed resulting in accumulating quantities of the autoinducer HSL. At a threshold concentration of HSL, correlating with a desired host cell density, expression of the gene of interest is activated through binding of the HSL/LuxR autoinducer complex to the luxI promoter.

In another aspect, the invention provides a host cell comprising a heterologous luxI gene integrated into the genome. In one embodiment of this aspect of the invention, the host cell also comprises luxR and further comprises an expression vector encoding a gene of interest operably linked to a promoter which responds to induction by the LuxR autoinducer complex. The luxR gene may be present in the vector or alternatively integrated into the genome of the host cell. In this manner, the host cell is configured such that expression of luxI results in the production of the autoinducer HSL, which positively regulates expression of the gene of interest when the autoinducer reaches a threshold concentration.

In yet a further aspect, the invention is a host cell comprising a heterologous luxR gene integrated into the genome. In one embodiment of this aspect of the invention, the host cell also comprises a luxI gene and further comprises a vector encoding a gene of interest operably linked to a first promoter which is responsive to induction by the LuxR autoinducer complex. The luxI gene may be present in the vector or alternatively integrated into the genome of the host cell. In this manner, the host cell is configured such that expression of LuxI results in the production of the autoinducer HSL, which is capable of positively regulating expression of the gene of interest when the autoinducer reaches a threshold concentration.

Integrating luxI in the genome of the host cell reduces the gene dosage of luxI to one copy per cell. As demonstrated in the Examples below, integrating luxI into the host cell genome has the effect of increasing the threshold cell density necessary for gene activation such that a higher level of cell growth can be obtained for optimal production of the recombinant protein. One way in which luxI genomic integration may convey this advantage is through allowing for a more slow and controllable accumulation of autoinducer. Having luxI in the genome means that the gene dose is controlled, thereby regulating production of HSL. When luxI is present in a vector on the other hand, high copy numbers of this gene can result in a more rapid production of HSL thereby lowering the threshold density necessary for autoinduction.

Another advantage is that the gene dosage of luxI and gene dosage of the gene of interest can be controlled independently such that a specific copy number of the luxI gene can be obtained on the one hand and a specific copy number on the other. In other embodiments, the copy number of a heterologous gene can be autonomously considered without affecting the regulation control of the expression system.

In other embodiments a host cell comprises a heterologous luxR gene stably integrated into the genome of the host cell. LuxR is a necessary element of the LuxR/HSL autoinducer complex and thus gene dosing of this gene is expected to result in the same advantages discussed above, when luxI is integrated into the genome.

In still other embodiments a host cell comprises both a heterologous luxR gene and a heterologous luxI gene, which are both stably integrated into the genome of the host cell.

“Stably integrated” as used herein means that the heterologous genes encoding LuxR-type and/or LuxI-type proteins are incorporated into the genomic DNA of the host cell and can be passed into daughter cells for at least several generations. Stable integration can be achieved by methods well known in the art. See Example 8.

Expression of Recombinant Proteins of Interest

Methods of producing proteins of interest according to the invention can be used on a small or large scale. Any of the host cells described above can be cultured under conditions which permit expression of the encoded proteins. For example, in one embodiment, the host cell comprises a vector comprising (i) a first heterologous gene of interest operably linked to a first promoter which is responsive to induction by the LuxR autoinducer complex; and (ii) an inducible second promoter driving expression of a second gene such that expression of the second gene interferes with expression of the heterologous gene of interest, wherein said host cell also comprises a heterologous luxI gene and a heterologous luxR gene. Prefereably, luxI and/or luxR are integrated into the genome, however both or one of these genes can alternatively be present in a vector within the host cell. Preferably, the process of host cell culture results in expression of the luxI and luxR genes which in turn results in the production of the LuxR autoinducer complex and activation of expression of the gene of interest when the autoinducer reaches a threshold concentration.

In another aspect, the invention provides a process as defined above further comprising (i) an inoculum phase of preparing an inoculum of the host cell under conditions which suppress expression of the gene of interest; and (ii) a culture phase wherein a host cell culture is prepared using the inoculum and wherein expression of the gene of interest is autoinduced during culture at a threshold level of cell density.

In one embodiment of this process the host cell comprises a vector comprising (i) a first heterologous gene of interest operably linked to a first promoter which is responsive to induction by the LuxR autoinducer complex; and (ii) an inducible second promoter driving expression of a second gene such that expression of the second gene interferes with expression of the heterologous gene, and wherein suppression of the gene of interest during the inoculum phase is attained by inducing activation of the inducible second promoter.

In another aspect, the invention is based on the realization that effective control of recombinant gene expression can be brought about through the implementation of a multi-phase process, wherein in the first phase, gene expression is effectively suppressed, even under conditions of high cell density, and wherein in the second phase, gene expression is triggered through autoinduction. More specifically, the inventors have established that by repressing gene expression in the first phase, a high density inoculum of host cells can be prepared without triggering recombinant protein production. Subsequently, through making use of the QS machinery described above, hosts cells can be cultured from the initial inoculum until an optimal cell density is reached, at which point, autoinduction of gene expression will occur resulting in production of the recombinant protein.

Large-Scale Production

In one embodiment the protein of interest can be produced by a large scale process using fermentation and an expression system of the invention. The host cells can be grown using a batch culture system in which the growth rate, nutrients, and metabolic concentrations can be modified during the growth process. In another embodiment, the host cells are grown using a fed-batch process in which the composition of the medium at the beginning of the process is defined and then nutrients are added as needed during the growth process. In another embodiment, the host cells are grown using a continuous process in which the culture is maintained in the exponential growth phase by the continuous addition of fresh medium that is balanced by the removal of cell suspension from the bioreactor

The overall culture process can be divided in several steps, including different phases (batch, fed-batch and carbon feed). For example, an overall culture process used for the recombinant protein production using a QS expression system of the invention can comprise the following phases: a) an initial phase of pre-inoculum, b) a phase of inoculum in which the bacteria start growing, c) a phase of expression of the protein of interest, d) a phase of harvesting the cells e) purification of the protein.
a. pre-inoculum phase. This phase allows the growth of bacteria at high density with the achievement of a very dense pre-inoculum with an OD from about 5-6 up to about 10 (e.g., 5, 5.5, 6, 6.6, 7, 8, 9, 10). During this phase, inhibition of synthesis of the protein of interest is recommended. The phase of pre-inoculum can be carried out for example in a batch system.
b. inoculum phase. The pre-inoculum is diluted in fresh medium, for example with a factor of dilution from 100 to 1000 (e.g., 100, 150, 200, 250, 300, 350, 400, 450, 500, 550, 600, 650, 700, 750, 800, 850, 900, 950, 1000). The volume of the fermentor is also increased. During this phase the bacteria start growing, and the protein expression should be inhibited. The inoculum phase can be carried out in a fed-batch system but is not limited by this method of culture. In some embodiments the fed-batch can be carried out by adding glucose from 1 g/l to 5 g/l (e.g., 1, 1.5, 2, 2.5, 3, 3.5, 4, 4.5, or 5 g/l).
c. expression phase. When the cell density reaches a desired certain optical density (e.g., 2-30D), growth conditions can be modified by changing the pH, due to exhaustion of some nutrients (e.g., glucose), and the expression of the protein can begin. The pH can be set, for example, from 6.2 to 7.8 (e.g., 6.2, 6.3, 6.4, 6.5, 6.6, 6.7, 6.8, 6.9, 7, 7.1, 7.2, 7.3, 7.4, 7.5, 7.6, 7.7, 7.8). The expression of the protein can take place from an OD of from 3 to 30 (e.g., 3, 4, 5, 6, 7, 8, 9, 10, 11, 12, 13, 14, 15, 16, 17, 18, 19, 20, 21, 22, 23, 24, 25, 26, 27, 28, 29, 30), which corresponds to a stationary phase.
   Inhibiting protein synthesis in an inoculum

One problem encountered using the pre-culture step in industrial fermentation in an auto-induction system is that the cellular density can be very high, which activates the auto-induction expression system of the invention, which produces the recombinant protein of interest. At this stage of the process, however, expression of the recombinant protein is not recommended because, for example, the protein of interest could compromise the vitality of the bacteria and the production of the protein in a bioreactor (Grossman et al., 1998. Gene 209 95-103). Because quorum sensing systems are induced by cell density, it is preferably to include a repression system in pre-culture phases. Thus, one embodiment of the invention includes the introduction of an accessory system which represses the auto-induction when cell density is high. This embodiment of the invention uses expression vectors described above which rely on transcriptional interference.

Another problem is the premature expression of the heterologous protein during the early stage of the growing phase (inoculum phase). Catabolic repression could be used to repress the auto-induction system. It is known that the presence of glucose in the medium of culture represses the fluorescence of the lux operon in E. coli (Dunlap and Kuo (1992) J. Bacteriol. 174:2440-8). Glucose can be added in fed-batch cultures during the early phase of growing, for example to a concentration ranging from 1 g/l to 5 g/l. In one embodiment, after exhaustion of glucose and at a sufficient cellular density, the heterologous protein can be expressed. The carbon source after consumption of the glucose can be, for example, glycerol, fructose, lactose, sucrose, maltodextrins, starch, inulin, vegetable oils such as soybean oil, hydrocarbons, alcohols such as methanol and ethanol, organic acids such as acetate, and molasses.

Following the culture (e.g., batch, feed-batch or continuous culture), further processing steps can be used to purify the protein of interest. Such methods are well known in the art and include size exclusion chromatography, ammonium sulfate fractionation, ion exchange chromatography, affinity chromatography, and preparative gel electrophoresis. A preparation of purified proteins of interest is at least 80% pure; preferably, the preparations are 90%, 95%, or 99% pure. Purity of the preparations can be assessed by any means known in the art, such as SDS-polyacrylamide gel electrophoresis.

Products

In another aspect, the invention provides a recombinant protein, vaccine or pharmaceutical composition obtained or obtainable by one or more of the methods disclosed above.

Pharmaceutical compositions of the invention will typically, in addition to the components mentioned above, comprise one or more “pharmaceutically acceptable carriers.” These include any carrier which does not itself induce the production of antibodies harmful to the individual receiving the composition. Suitable carriers typically are large, slowly metabolized macromolecules such as proteins, polysaccharides, polylactic acids, polyglycolic acids, polymeric amino acids, amino acid copolymers, and lipid aggregates (such as oil droplets or liposomes). Such carriers are well known to those of ordinary skill in the art. A composition may also contain a diluent, such as water, saline, glycerol, etc. Additionally, an auxiliary substance, such as a wetting or emulsifying agent, pH buffering substance, and the like, may be present. A thorough discussion of pharmaceutically acceptable components is available in Gennaro (2000) Remington: The Science and Practice of Pharmacy. 20th ed., ISBN: 0683306472.

Vaccine compositions of the invention may be administered in conjunction with other immunoregulatory agents. In particular, compositions will usually include an adjuvant. Adjuvants for use with the invention include, but are not limited to, mineral containing compositions (e.g., mineral salts, such as aluminum salts and calcium salts), oil-emulsions (e.g., MF59 (5% Squalene, 0.5% TWEEN™ 80, and 0.5% Span 85, formulated into submicron particles using a microfluidizer), saponin formulations (e.g., QS21 and ISCOMs), virosomes and virus like particles (VLPs), bacterial or microbial derivatives (e.g., non-toxic derivatives of enterobacterial lipopolysaccharide, lipid A derivatives, immunostimulatory oligonucleotides, ADP-ribosylating toxins and detoxified derivatives thereof, bioadhesives and mucoadhesives, microparticles, liposomes, polyoxyethylene ether and polyoxyethylene ester formulations, polyphosphazene (PCPP), muramyl peptides, imidazoquinoline compounds, thiosemicarbazone compounds, and tryptanthrin compounds, and immunomodulators such as IL-1, IL-2, IL-4, IL-5, IL-6, IL-7, IL-12, interferons such as interferon-γ, macrophage colony stimulating factor, and tumor necrosis factor.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### Example 1

### Host Cell Strains and Growth Conditions

Features of bacterial strains used in these Examples are summarized in Table 4.

TABLE 4

| Strain | Features |
|---|---|
| V. fischeri (ATCC | |
| 7744) | |
| E. coli DH5α | F⁻Φ80lacZΔM15Δ(lacZYA-argF)U169 |
| | deoR recA1 endA1 hsdR17 (r⁻ₖ, M⁻ₖ) |
| | phoA supE44 thi-1 gyrA96 relA1 λ⁻ |
| E. coli TOP10 | F⁻mcrA Δ(mrr-hsdRMS-mcrBC) |
| | Φ80lacZΔM15 ΔlacX74 recA1 araD139 Δ(ara- |
| | leu) 7697 galU galK rpsL (Str^{r}) endA1 nupG |
| E. coli MM294.1 | F⁻ supE44 hsdR17 endA1 thi-1 lambda⁻ |
| (ATCC 39515) | |
| E. coli MM294.1::luxI | F⁻ supE44 hsdR17 endA1 thi-1 lambda⁻ |
| | metW::ery\luxI |
| BL21(DE3) | F⁻ ompT gal dcm lon hsdS_{B}(r_{B}⁻ m_{B}⁻) 1(DE3 |
| | [lacI lacUV5-T7 gene 1 ind1 sam7 nin5]) |
| E. coli HK 100 | F⁻ mcrA (mrr-hsdRMS-mcrBC) 80lacZ M15 |
| | lacX74 recA1 endA1 araD139 (ara- |
| | leu)7697 galU galK rpsL(Str^{R}) nupG + |
| | additional uncharacterized resistance to |
| | bacteriophage(s) |

E. coli strains were grown in liquid and solid medium. Solid medium was obtained by adding 1.5% agar to liquid medium. The following liquid media were used: LB (1% Bactotriptone, 0.5% yeast extract, 0.5% NaCl), YE3× (45 g/l yeast extract, 4 g/l KH₂PO₄, 16 g/ K₂HPO₄, 15 g/l glycerol), minimal medium 3 g/l (NH₄)₂SO₄, 1 mM MgSO₄, 1 mM thiamin, 1 mM FeSO₄, MnCl₂, CoCl₂, CaCl₂, CuSO₄, ZnSO₄, 4 g/l, KH₂PO₄, 16 g/l K₂HPO₄, 15 g/l glycerol), HTMC (30 g/l Yeast Extract, 16 g/l K₂HPO₄, 4 g/l KH₂PO₄, 15 g/l Glycerol). Strains were grown at 25° C., 27° C., 30° C., or 37° C.

V. fischeri were grown in the LBS medium containing 10 g of Bacto Tryptone-Peptone, 5 g yeast extract, 50 ml of 1 M Tris base (Sigma Chemical Co., St. Louis, Mo.) at pH 7.5, and 20 g/l NaCl at 28° C. (McCann et al., 2003, Appl Environ Microbiol 69:5928-34).

Batch Fermentation

The medium used for the batch fermentation was YE3x (45 g/l Yeast extract, 4 g/l KH₂PO₄, 16 g/ K₂HPO₄, 15 g/l glycerol), minimal medium 3 g/l (NH₄)₂SO₄, 1 mM MgSO₄, 1 mM thiamin, 1 mM FeSO₄, MnCl₂, CoCl₂, CaCl₂, CuSO₄, ZnSO₄, 4 g/l, KH₂PO₄, 16 g/l K₂HPO₄, 15 g/l glycerol). Antibiotics (e.g., ampicillin or kanamycin) were added to the medium as needed. The growth phase was carried out at 25° C. and at pH of 6.2 or 7.2 (±0.1). Dissolved oxygen concentration was maintained above the setpoint of 40%. Air was supplied at a fixed rate of 0.5 VVM (volume of gas/volume of liquid×minutes). When the dissolved oxygen value dropped down to the setpoint value, the minimal concentration (40%) was maintained by controlling in cascade agitation rate from 200 to 800 rpm and successively addition of molecular oxygen from 0-0.05 VVM.

Cellular growth was monitored by reading the optical density at 590 nm (Beckman spectrophotometer) of samples taken every hour. These samples were used for several analyses. The production of recombinant proteins was achieved in bioreactors of 2 and 5 liters of working volume (Applikon Analytical B. V., A c Schiedam, Netherlands). The agitation, pH, oxygenation and temperature parameters were followed using ADI 1030 (Applikon) and the Bioexpert software (Applikon) for a continuous monitoring of these parameters.

Fluorescence Measurements

Fluorescence of the bacterial cultures was monitored using fluorescence activated cell sorting (FACS) and by plate reader fluorimeter (Infinite M200-Tecan).

FACS Assay. Samples were taken at various points in the growth phase for fluorescence analysis by FACS (LSR-II, Becton-Dickinson). Samples were standardized to an OD of 0.5 by centrigation at 6000×g for 5 minutes. After two washings in PBS, pellets were resuspended in the same volume of PBS with 2% formaldehyde. Samples were incubated 20 minutes at room temperature, then washed two times with PBS and stored at 4° C. until FACS analysis.

Five- to ten-thousand events were acquired for each sample. The results were analyzed via the use of the FlowJo computer software (Tree Star). The negative control was BL21(DE3)\pET21b E. coli culture. The positive control was E. coli BL21(DE3)\pET21b-gfp E. coli culture (see Tables 3 and 4 for strain and vector features). Both E. coli control strains were grown under each of the experimental conditions used and induced with 1 mM IPTG at the optical density of 0.5 at 590 nm. Cells were harvested two hours after induction.

Fluorimeter Assay on Microtiterplates

Fluorescence was also measured during growth of the cultures using the microplate Infinite M200 (Tecan). The concentrations of the E. coli strains were normalized by growing them overnight. The assays were carried out at least in triplicate and standardized at an optical density of 0.01 at 590 nm in M9, LB, and YE3X mediums in presence of antibiotics. Temperature and agitation were controlled during the growth, and the fluorescence was measured in-line with an excitation wave at 460 nm, an emission wave at 510 nm, and the absorbance at 590 nm.

### Example 2

### Plasmid Construction

Features of plasmids are described in Table 5.

TABLE 5

| Plasmid | Features |
|---|---|
| pGlow TOPO | TA cloning vector for PCR products; Neo^{r} Ap^{r} |
| | pBR322 origin, f1 origin, T7 promoter, gfp orf |
| pGlow TOPO+ | pGlow with metE gene interrupted by Ery^{r} |
| metE::ery | |
| pGLlux506 | pGlow with luxR-luxI PCR fragment and with |
| | gfp in orf with P_{luxI.} |
| pCRII | TA cloning vector for PCR products, KanR, |
| | AmpR. pUC Origin, f1 origin, lacZα |
| pCRII MHTT | pCRII-MHTT fragment |
| pCR-MT | pCRII-MT fragment |
| pCRII-MluI- | pCRII-luxR_{TTA}\luxC_{ATG} fragment with MluI and |
| luxR_{TTA}\luxC_{ATG}- | AatII restriction sites. |
| AatII | |
| pLAIR32 | pCRII-MT-luxR-luxI fragment |
| pLAIR42 | pCRII-MHTT-luxR-luxI fragment |
| pLAIET32 | Ori pBR322 Amp^{r}-MT-luxR-luxI fragment |
| pMKSal | Ori pBR322, Kan^{r}, T7 promotor, luxI- |
| | optimized, luxR-optimized. |
| pMKSal-GFP | pMKSal, gfp in orf with P_{luxI} |
| pMKSal ΔG-3526 | pMKSal, ΔG-3526 under the control of P_{luxI} |
| pMKSal-ΔluxI- | pMKSal with truncated luxI using KpnI sites |
| pMKSal-ΔluxI-GFP | pMKSal-ΔluxI-; gfp in orf with P_{luxI.} |
| pKOBEG | Expression of the genes gam, bet and exo for |
| | allowing homologous recombination |
| pGLEM | Neo^{r} Ap^{r}, pBR322 origin, f1 origin, T7 |
| | promotor |
| pGLEM-luxI | gfp orf, metE gene interrupted by Ery^{r}, |
| | pGLEM-luxI in AscI site |
| pET21 | |
| pET21-GFP | pET21 with gfp in orf with p7 promoter |

gfp gene amplification. The gfp gene was amplified from pGlow (Invitrogen) using a mixture of GFPEcoRI/GFPNotI primers (see Table 6).

TABLE 6

| Primer | Sequence | SEQ ID NO: |
|---|---|---|
| LuxFr | AAGCTTTACTTACGTACTTAACTTTTA | 98 |
| LuxRv | TCATTATTTCCCCTATAATATACTTAGT | 99 |
| LFrMluIT | TTTTACGCGTTACTTACGTACTTAACTTTTA | 100 |
| LRvAatIIT | TTTTGACGTCTTCATTATTTCCCCTATAATATA | 101 |
| pETORIAMPMluIFr | TTTTACGCGTGAGAAGCAGGCCATTATCGC | 102 |
| pETORIAMPNdeIRv | TTTTCATATGATTTCAGGTGGCACTTTTCG | 103 |
| GFPEcoRI | GAATTCAATGGCTAGCAAAGGAGAAGAACT | 104 |
| GFPNotI | GCGGCCGCTTATTTGTAGAGCTCATCCA | 105 |
| GFPEcoRII | GAATTCATGGCTAGCAAAGGAGAAGAAC | 106 |
| LxIAscIF | TTTTGGCGCGCCCATTATTTCCCCTATAATATACTTAGTA | 107 |
| LxIAscIR | TTTTGGCGCGCCTAAAACGGTAATAGATTGACA | 108 |
| metEL | GAAAAGTACTGCTTGTAGCGTTTTCAGGTG | 109 |
| LuxRv | GAAAAGTACTGGGAAGAAGTCGCTGTAATG | 110 |
| LFrMluIT | TCAAATGTCAATCTATTACCG | 111 |
| LRvAatIIT | TCCTTACCTATTGTTTGTCG | 112 |

The fragment was digested by EcoRI and NotI restriction enzymes and were cloned in different plasmids such as pMKSa1, pMKSa1-ΔluxI, pET21 which are described in Table 5.

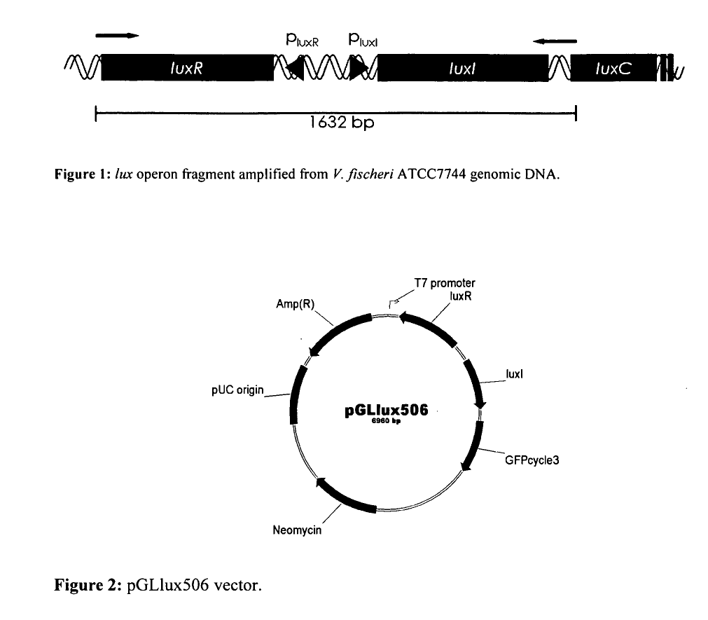

pGLlux506. The fragment luxR_{TTA}\luxC_{ATG} of lux operon (FIG. 1) which comprises the gene luxR, luxI, intergenic region between luxR-luxI, and the region luxI-luxC was amplified from V. fischeri ATCC7744 genome using a mixture of LuxFr\LuxRv primers (Table 6). The fragment luxR_{TTA}\luxC_{ATG} contains ATG codon of luxC gene which is in frame with gfp reporter gene when introduced at the TA site in the pGLOW vector (Invitrogen). This new vector is called pGLlux506. E. coli MM294.1 is transformed with pGLlux506 vector. The expression of gfp gene is under the control of the luxI promoter (FIG. 2), which means the expression of Gfp protein is dependent on cell density.

Fluorescence, as measured by FACS (infinite M200-Tecan) of cells at different growth stages, was evidenced only when the OD reached a threshold level. The induction of the synthesis of Gfp is observed between an optical density between 0.5 and 0.7. This result indicates that a large quantity of protein can be produced by this autoinduction system.

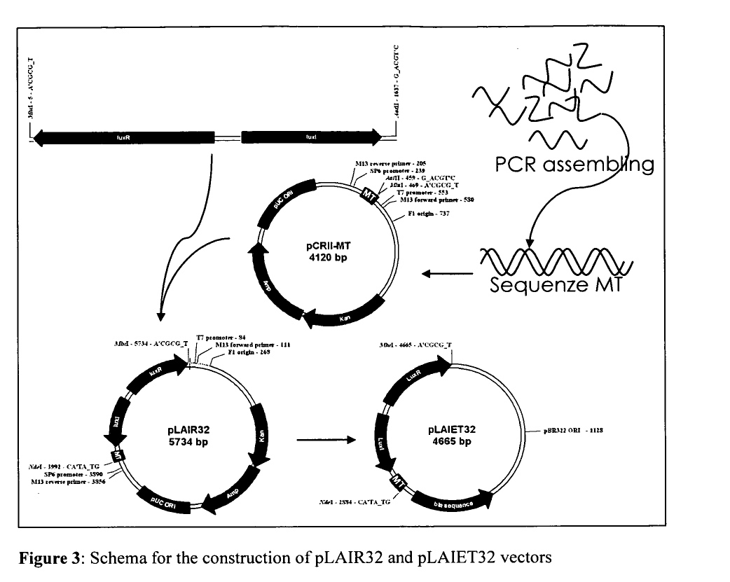

pLAIET32 vector. The construction of the pLAIET32 vector is summarized in FIG. 3.

The MT fragment were assembled using assembling PCR as described in Rydzanicz et al. (2005, Nucleic Acids Research, 33:W521-W525) and using the “Assembly PCR oligo maker” program, accessible on following interne site (publish.yorku.ca/˜pjohnson/AssemblyPCRoligomaker.html). The designed MT sequence was inserted in the program with the following parameters: monovalent cation concentration (50 mM), DNA concentration (0.5 μM), maximum oligonucleotide length calculated (50), annealing temperature (55° C.), acceptable melting temperature for overlapping (40° C.). Then the sequences of the different primers for assembling PCR and for the full length PCT were given (see Table 7).

Apo R and Apo F are the flanking primers and Apo 1-6 are the assembly oligonucleotides.

TABLE 7

| Primer | Sequence | SEQ ID NO |
|---|---|---|
| Apo1 | TTTTAAGCTTACGCGTGGTACCAAGACGTCGAAG | 113 |

| | | |
|---|---|---|
| Apo2 | GGCCGCTCGAGTCGACGGAATTCTTCGACGTCTTGGTACCA | 114 |
| Apo3 | GTCGACTCGAGCGGCCGCGTGACTGACTGAAGATCTACTAGTAG | 115 |
| Apo4 | CTGAGCCTTTCGTTTTATTTGAAAGCTACTAGTAGATCTTCAGTCAGTC | 116 |
| Apo5 | CTTTCAAATAAAACGAAAGGCTCAGTGCAAAGACTGGGCCTTTCGT | 117 |
| Apo6 | AAAACATATGCAGATTAAAACGAAAGGCCCAGTCTTTGC | 118 |
| ApoF | TTTTAAGCTTACGCGTGGTACC | 119 |
| ApoR | AAAACATATGCAGATTAAAACGAAAGGC | 120 |

The MT fragment obtained by PCR assembling was inserted in the TA sites in the plasmid (Invitrogen) (FIG. 3) and the new vector was called pCR₁₁-MT. In parallel, the luxR_{TTA}\luxC_{ATG} fragment was subcloned in the vector pCR₁₁ using the LFrMluIT\LRvAatII primers (Table 6) for obtaining the pCR₁₁-MluIluxR_{TTA}\luxC_{ATG}AatII vector. The vector pCR₁₁-MT was obtained.

The fragment which contains the pBR₃₂₂ on and the bla gene for the resistance to ampicillin was amplified with pETORIAMPMluIFr\pETORIAMPNdeIRv primers from pET21 plasmid. The PCR product was digested by MluI\NdeI restriction enzymes and ligated with the MluI\NdeI fragment from pLAIR32 plasmid, to get the pLAIET32 plasmid.

The same protocol was used for the fragment MHTT which differs from the MT fragment by the presence of a His-Tag and the presence in the sequence of a site of cleavage for the Tev-protease. The sequences of the different primers for assembling PCR and for the full length PCT were given (see Table 8). ApoFRv and ApoFFr are the flanking primers and Apom 1-6 are the assembly oligonucleotides. The vectors were named pLAIR42 and pLAIET42.

TABLE 8

| Primer | Sequence | SEQ ID NO |
|---|---|---|
| Apom1 | TTTTAAGCTTACGCGTGGTACCAAGACGTCGCATCATCATCA | 121 |
| Apom2 | CTTGCCCTGGAAGTACAGGTTTTCGTGATGATGATGATGATGCGACGTCTTGG | 122 |
| Apom3 | AACCTGTACTTCCAGGGCAAGAATTCCGTCGACTCGAGCGGCCGCGTG | 123 |
| Apom4 | TTCGTTTTATTTGAAAGCTACTAGTAGATCTTCAGTCAGTCACGCGGCCGCTC | 124 |
| Apom5 | AAGATCTACTAGTAGCTTTCAAATAAAACGAAAGGCTCAGTGCAAAGACTGGGCCT | 125 |
| Apom6 | AAAACATATGCAGATTAAAACGAAAGGCCCAGTCTTTGCACT | 126 |
| ApomFFr | TTTTAAGCTTACGCGTGGTACC | 127 |
| ApomFRv | AAAACATATGCAGATTAAAACGAAAGGC | 128 |

### Example 3

### Repression of the Auto-Induction System at High Cellular Density

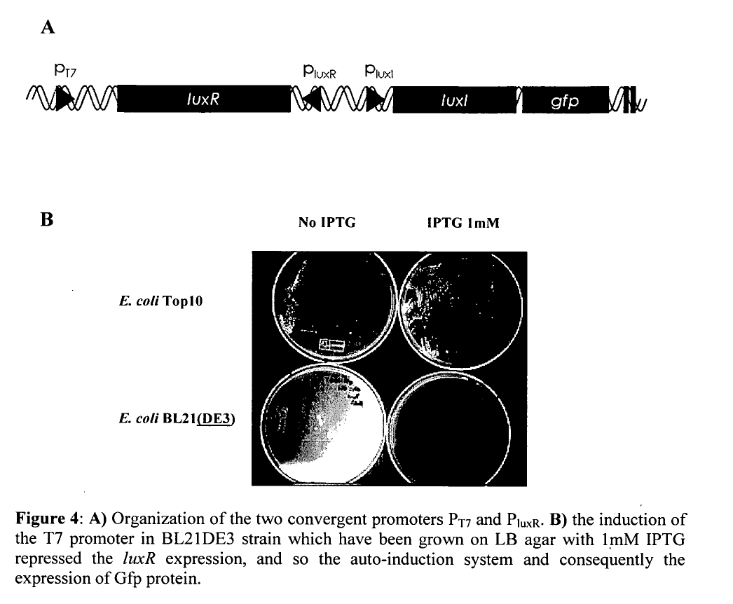

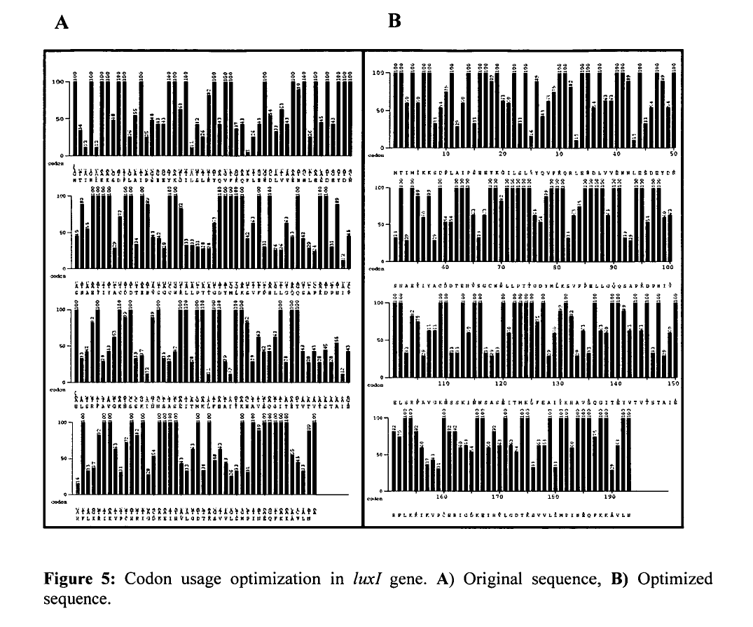

The inhibition of the auto-induction system at high cell density was obtained by inhibiting the expression of luxR. For this purpose, an inducible promoter (here, the T7 promoter) was inserted upstream of the luxR gene and in a convergent orientation to the promoter of luxR gene (FIG. 4A) in the auto-inducible expression vector. This vector was introduced in the E. coli BL21DE3 strain, where the T7 promoter was inducible by the addition of IPTG in the medium and the repression of luxR by transcriptional interference was tested.

The BL21DE3 and the negative control E. coli Top 10 strains (Table 4) were grown on LB agar in absence or presence of 1 mM IPTG. BL21DE3 cells in absence of IPTG showed a strong fluorescence whereas in presence of IPTG the level of fluorescence was the same as in the negative control. The induction of the T7 promoter in BL21DE3 strains which have been grown on LB agar with 1 mM IPTG repressed the luxR expression; therefore the auto-induction system and consequently the expression of Gfp protein also was repressed (FIG. 4B). The luxR repression system via transcriptional interference was found to be tunable and suitable for the repression of the auto-induction expression system at high cellular density.

### Example 4

### Sequence Optimization of luxR and luxI for the Expression in E. Coli

Optimization of luxI and luxR sequences for the expression in E. coli. The sequences of luxI and luxR were optimized in order to increase the expression of the QS machinery. Vecton NTI analysis of the nucleotide sequence has showed that the translation of luxR and luxI mRNA requires the use of rare tRNA in E. coli. The amino acid sequence of LuxI and LuxR proteins were reverse translated using Table 9.

TABLE 9

| Amino | |
|---|---|
| Acid | Codon |
| Ala (A) | GCT |
| Cys (C) | TGC |
| Asp (D) | GAC |
| Glu (E) | GAA |
| Phe (F) | TTC |
| Gly (G) | GGT |
| His (H) | CAC |
| Ile (I) | ATC |
| Lys (K) | AAA |
| Leu (L) | CTG |
| Met (M) | ATG |
| Asn (N) | AAC |
| Pro (P) | CCG |
| Gln (Q) | CAG |
| Arg (R) | CGT |
| Ser (S) | TCT |
| Thr (T) | ACC |
| Val (V) | GTT |
| Trp (W) | TGG |
| Tyr (Y) | TAC |
| Stop (*) | TGA |

The optimized luxI sequence (SEQ ID NO:134) (FIG. 5B) has several codon modifications and is 74.742% identical to the original sequence of luxI (FIG. 5A). The GC content of the original sequence was 32%, and that of the optimized sequence is 45%. Two enzymatic restriction sites, for KpnI and XbaI, were introduced.

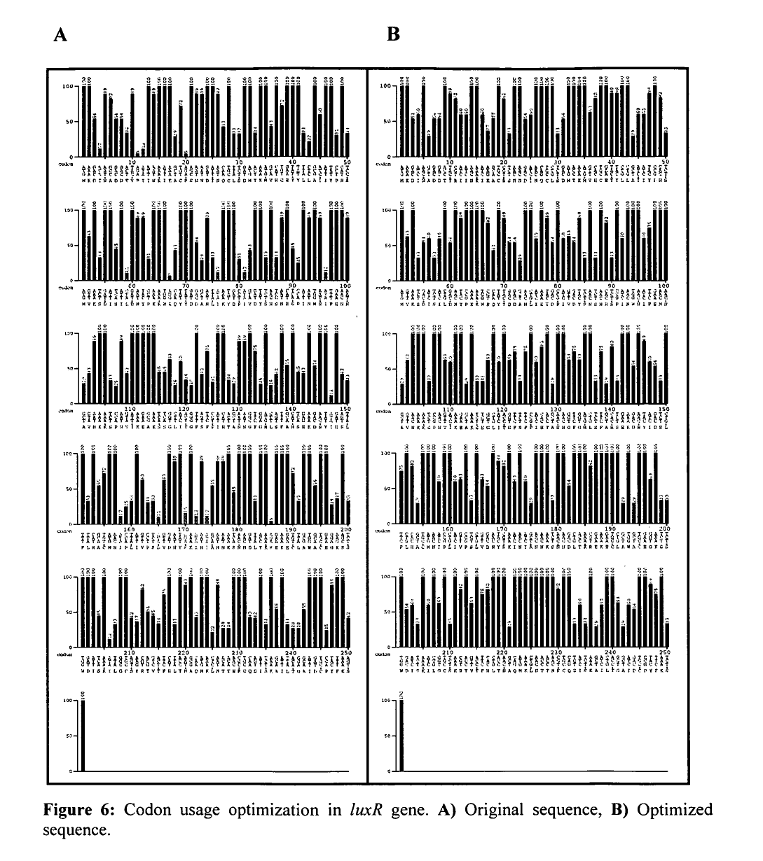

Similarly, the optimized luxR sequence (SEQ ID NO:133) (FIG. 6B) has several codon modifications and is 74,235% identical to the original sequence of luxR (FIG. 6A). The GC content of the original sequence was 30%, and that of the optimized sequence is 45%.

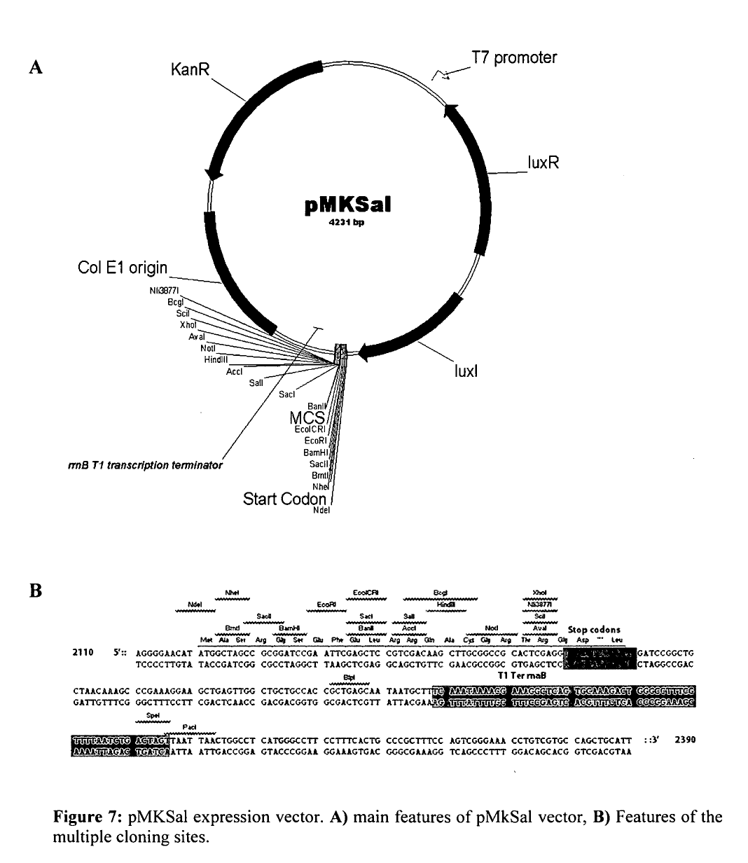

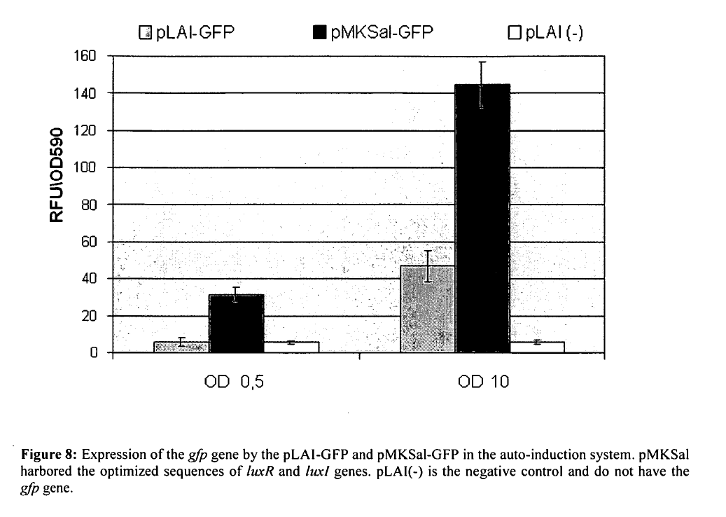

Construction of pMKSal vector. A DNA fragment containing the T7 promoter, the optimized luxR gene, the luxR-luxI intergenic region, the optimized luxI gene, a multiple cloning site (MCS), and a transcription terminator was designed and synthesized. This fragment was inserted into a pMK vector, which has kanamycin-resistance gene, using AscI and PacI cloning sites. This resulted in a new vector, pMKSal (FIG. 7A). pMKSal has an origin of replication ColE1. The MCS is derived from the MCS of the pET21a plasmid, which is compatible for the cloning using different expression vectors (FIG. 7B). The promoter T7 is convergently oriented with respect to luxR. The sequences of luxR and luxI are the optimized sequences of luxR and luxI. The intergenic region between the two gene luxR and luxI was not modified. The MCS is downstream the luxI gene and permits the insertion of the heterologous gene of interest for the production of the recombinant protein. The gfp gene (SEQ ID NO:150) was inserted in the MCS of pMKSal as described above and is called pKMSal-GFP. The gfp gene is under the control of the LuxR/autoinducer induced promoter.

The pMKSaI-GFP shows a high expression level compare to the vector which harboured the native luxR ((SEQ ID NO:77) and luxI (SEQ ID NO:76) sequences with pLAIET32. Fluorescence was measured at two different cell concentrations, corresponding to the pre-induction stage (OD=0.5 at 590 nm) and the induction stage (OD=10 at 590 nm), respectively. The level of fluorescence, which reflects synthesis of the Gfp, is 2.5 higher than the expression of the pLAIET32-GFP (FIG. 8). The pLAIET32-GFP and the pMKSal-GFP are considered to be equivalent vectors. They have the same origin of replication, the size of the vector is approximatively the same (pMKSal 4231 bp, pLAIET32 4665). These vectors differ by the antibiotic resistance and their MCS.

The pMKSal vector harboring the optimized luxR and luxI genes was demonstrated to be a very efficient vector for the production of recombinant proteins here the Gfp protein. It provides a simplified cloning approach and improved the efficiency for the expression of target gene.

Via the presence of the T7 promoter on pMKsal, the expression of luxR gene can also be repressed and therefore used to regulate the quorum sensing machinery and the transcription of the target gene in high cell density growth conditions.

### Example 5

### Synthesis of Gfp Protein

The overall culture process for the expression of Gfp protein included the following. E. coli MM294.1/pMKSa1-gfp strain was grown in a volume of 50 ml in batch culture in Ye3X medium completed with 100 ng/microliter of Kanamycin, IPTG 1 mM and 5 g/l glucose, at 25° C., at 7.2 pH, with agitation at 180 rpm. The cells (pre-inoculum) were grown until an optical density of 5 at 590 nm. Then the pre-inoculum (50 ml) was diluted in 5000 ml of Ye3X medium completed with 100 ng/microliter of Kanamycin and glycerol (10 g/l). Cells were grown at 25° C. with agitation at 180 rpm at pH 7.8 in a fed-batch culture with a feed of 1 g/l glucose until a optical density of 3 at 590 nm was reached. At this optical density, the pH was switched to 6.2 and the glucose feed was stopped. The synthesis of Gfp was auto-induced at about this cellular density, and cells were grown until the stationary phase was reached. The cells were then collected, and the synthesis of Gfp was assessed by measuring the fluorescence as previously described.

### Example 6

### Amplification of the Expression System Response to Different Cell Density

To amplify the response of the expression system at different cell density, a library of mutant luxR genes was constructed using an Error Prone Polymerase Chain Reaction (EP-PCR) Random Mutagenesis as described in Cadwell & Joyce (1992). EP-PCR is a PCR in which the fidelity of the Taq polymerase is decreased without significantly reducing the level of amplification accomplished in the PCR. This can be achieved by modifying the concentration of MgCl₂, by addition of MnCI₂ to the reaction mixture, and use of unbalancing concentrations of the four dNTPs.

Tables 10 and 11 summarize the reaction conditions used in this example.

TABLE 10

| | Stand | | Mut 1 | | Mut 2 | |
|---|---|---|---|---|---|---|
| pGL506lux | 2 | ng | 2 | ng | 2 | ng |
| Buffer 10x | 10 | micl | 10 | micl | 10 | micl |
| LuxFr | 30 | pmoles | 30 | pmoles | 30 | pmoles |
| LuxRv | 30 | pmoles | 30 | pmoles | 30 | pmoles |
| MgCl2 | 1.5 | mM | 7 | mM | 7 | mM |
| dATP | 0.2 | mM | 0.2 | mM | 0.2 | mM |
| dTTP | 0.2 | mM | 1 | mM | 1 | mM |
| dCTP | 0.2 | mM | 1 | mM | 1 | mM |
| dGTP | 0.2 | mM | 1 | mM | 0.2 | mM |
| MnCl2 | — | | 0.5 | mM | 0.5 | mM |
| Tac pol Platinum | 2.5 | U | 5 | U | 5 | U |

| | | | |
|---|---|---|---|
| H2O to 100 micl | 64.4 | 23.9 | 31.9 |

TABLE 11

| | |
|---|---|
| 94° C. × 2′ | X1 |
| 94° C. × 30″ | X30 |
| 53° C. × 30″ | |
| 72° C. × 2′ | |
| 72° C. × 2′ | X1 |
| 4° C. | Xω |

EP-PCR was used to amplify the fragment of lux operons which comprised the luxR gene, the luxI gene, the intergenic region between luxR and luxI, and the region luxI-luxC up to the start codon of the luxC gene (SEQ ID NO:130) with a mixture of LuxFr and LuxRv primers (Table 6). The amplified fragment was cloned in the TA site of the pGLOW-topo (Invitrogen, Table 5) in frame with the gfp gene.

All fluorescent colonies were selected and inoculated in fresh medium containing antibiotics, and growth was normalized overnight. The ability of these selected colonies to express the gfp gene was tested by monitoring real-time fluorescence microtiter fermentation. Eighty-eight clones that expressed the gfp by Quorum Sensing behavior were obtained. Sequence analysis showed that all the mutations were localized in the luxR gene. Features of the different mutants are summarized in Table 12, in which the amino acid positions are numbered according to SEQ ID NO:42. The nucleotides are numbered according to the coding sequence for wild-type LuxR, SEQ ID NO:77.

TABLE 12

| | Mutation | Nucleotide | Initial amino acid | Resulting amino acids | |
|---|---|---|---|---|---|
| Mutant(s) | type | sequence | sequence | sequence | Codon sequence |
| 1M37-2M27- | 1) silent | nt 57 T→C | LuxR: -Ala-Cys(19)-Arg- | -Ala-Cys(19)-Arg- | TGT(Cys-5.1%) → TGC (Cys-6.4%) |
| 2M28 | 2) deletion | del 748 A | LuxR: -Lys-Ser(250)-STOP | -Lys-Val(250)-Lys-Tyr- | AGT (Ser 8.7%)→ GTT (Val 18.3) |
| | | | | Val-Ser-Lys-Ala-Stop | |
| 1M6 | 1) silent | nt 493 C→T | LuxR: -Ser-Leu(165)-Val- | -Ser-Leu(165)-Val- | CTA (Leu-3.1%) →TTA (Leu-10.1%) |
| | 2) silent | nt 57 T→C | LuxR: -Ala-Cys(19)-Arg- | -Ala-Cys(19)-Arg- | TGT (Cys-5.1%) → TCG (Cys-6.4%) |
| 1M8-1M9-1M14- | 1) silent | nt 57 T→C | LuxR: -Ala-Cys(19)-Arg- | -Ala-Cys(19)-Arg- | TGT (Cys-5.1%) → TCG (Cys-6.4%) |
| 1M21-1M24- | | | | | |
| 1m29-2M17 | | | | | |
| 1M16 | 1) silent | nt 57 T→C | LuxR: -Ala-Cys(19)-Arg- | -Ala-Cys(19)-Arg- | TGT (Cys-5.1%) → TCG (Cys-6.4%) |
| | 2) missense | nt 22 G→A | LuxR: -Asp-Asp(8)-Thr- | -Asp-Asn(8)-Thr- | GAC (Asp-37.2%) → AAC (Asn-59.13%) |
| 1M27 | 1) silent | nt 57 T→C | LuxR: -Ala-Cys(19)-Arg- | -Ala-Cys(19)-Arg- | TGT (Cys-5.1%) → TCG (Cys-6.4%) |
| | 2) nonsense | nt 745 A →T | LuxR: -Phe-Lys(249)-Ser- | -Phe-STOP | AAA (Lys 75.44) → TAA (Stop codon) |
| 2M15 | 1) silent | nt 57 T→C | LuxR: -Ala-Cys(19)-Arg- | -Ala-Cys(19)-Arg- | TGT(Cys-5.1%) → TCG (Cys-6.4%) |
| | 2) deletion | del 748 A | LuxR: -Lys-Ser(250)-STOP | -Lys-Val(250)-Lys-Tyr- | AGT (Ser 8.7%)→ GTT (Val 18.3) |
| | 3) deletion | del 770 T | LuxR: -Lys-Ser(250)-STOP | Val-Ser-Lys-Lys-Ala- | TAA (Stop codon) →AAG (Lys 10.3%) |
| | | | | Lys-Gly-Asn-Ser- | |
| | | | | Thr-Thr-Leu-Asp-STOP | |

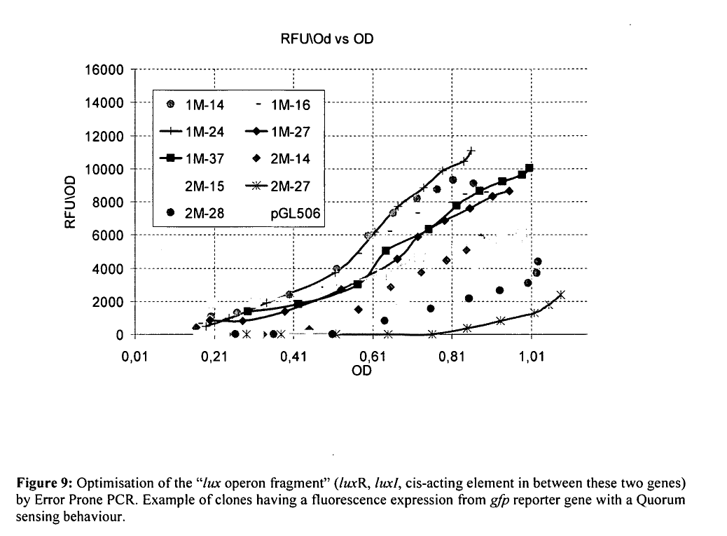

Clones with a low basal expression at a low cell density compared to the control MM294.1\pGLlux506 and which had an increased fluorescence in induced condition were selected (FIG. 9). The pattern of expression can be defined by the moment of induction, maximum expression level and all its intermediate phases with characteristic kinetic behavior. The selected mutants constitute a panel of expression systems with intrinsic differences in expression regulation and strength.

### Example 7

### Reduction of luxI Dosage by Chromosomal Integration

Integration of luxI gene in E. coli MM294.1 genome. The reduction of gene dosage of luxI by chromosomal integration was used to obtain a low basal expression level and a strong induction with tight control of gene expression. A copy of the wild-type luxI gene (SEQ ID NO:76) was stably integrated in the E. coli MM294.1 genome by homologous recombination using the helper plasmid pKOBEG. The pKOBEG plasmid is a thermosensitive replicon in which is present the λ phage redyβα operon expressed under the control of the arabinose-inducible pBAD promoter. pKOBEG is derived from the medium copy number plasmid pSC101, known to be maintained very stably in E. coli strains. It confers chloramphenicol resistance, so it can be transmitted in E. coli strains (Chaveroche et al., 2000). This system strongly promotes homologous recombination in E. coli. Its features are described in Table 5. X gam, bet and exo gene products encode an efficient homologous recombination system. The Gam protein is able to inhibit the Exonuclease V activity of RecBCD permitting the transformation of linear DNA (Unger et al., 1972; Unger and Clark, 1972). The bet and exo gene products are able to promote homologous recombination at short regions of homology between the PCR product and the chromosome.

The wild type luxI gene (SEQ ID NO:76) was amplified from pGLlux506 using the LxIAscIF\LxIAscIR primers (Table 6). The fragment was digested with AscI restriction enzyme and inserted in the pGLEM vector at the AscI restriction site. This new vector was called pGLEM-luxI. This plasmid contains the metE gene (ΔmetE) which is interrupted by the gene for the resistance to erythromycin and by the luxI gene. The fragment ΔmetE-erm-luxI-ΔmetE was amplified from the pGLEM-luxI using the primers metEL/metER (Table 6).

E. coli MM294.1 cells which contain the pKOBEG plasmid, were made competent for the uptake of the fragment and for the homologous recombination. To render the cells competent, they were grown in LB medium overnight at 30° C. When the optical density reached 0.2, the inducer L arabinose was added to a final concentration of 0.2% for the induction of the gam, bet and exo genes. The cells were grown until the culture reached an optical density of 1.

Competent E. coli MM294.1/pKOBEG cells were transformed with 1 microgram of the fragment ΔmetE-erm-luxI-ΔmetE. After transformation, 50, 100, or 150 microliters of the culture were seeded in Petri dishes containing LB with 100 μg/ml erythromycin and in Petri dishes containing LB and 40 μg/ml kanamycin. After incubation at 37° C. overnight, colonies were grown on Petri dishes containing LB and 20 μg/ml chloramphenicol and then on LB and 100 μg/ml erythromycin or on LB and 40 μg/mm kananamycin overnight at 30° C. to be sure than the clones had lost the helper plasmid. Then clones were grown at 37° C. for several passages until they lost the helper plasmid pKOBEG.

The clones which were resistant to erythromycin and sensitive to ampicillin and chloramphenicol were tested. The luxI cassette in the MM294.1 genomic DNA is schematized in the FIG. 10C. The integration of the luxI gene by homologous recombination in the metE locus in the E. coli MM294.1 genome was confirmed by PCR using the LuxI5\LuxI6 primers and Southern blot (FIG. 10B). The new strain was called MM294.1::/luxI. For the Southern blot, the probe was labeled using the “Amersham ECL direct Nucleic Acid Labelling and detection system.” The probe was obtained by PCR using the LuxI5\LuxI6 primers which amplified a nucleotide sequence of luxI of about 500 bases (FIG. 10A). Genomic DNA from the selected cloned was digested by XmaI and AatII restriction enzymes. The positive control was the product of the PCR amplified with LuxI5\LuxI6 and the pGLLux506 plasmid DNA after digestion by XmaI and AatII restriction enzymes. The negative control was genomic DNA of the E. coli MM294.1 strain.

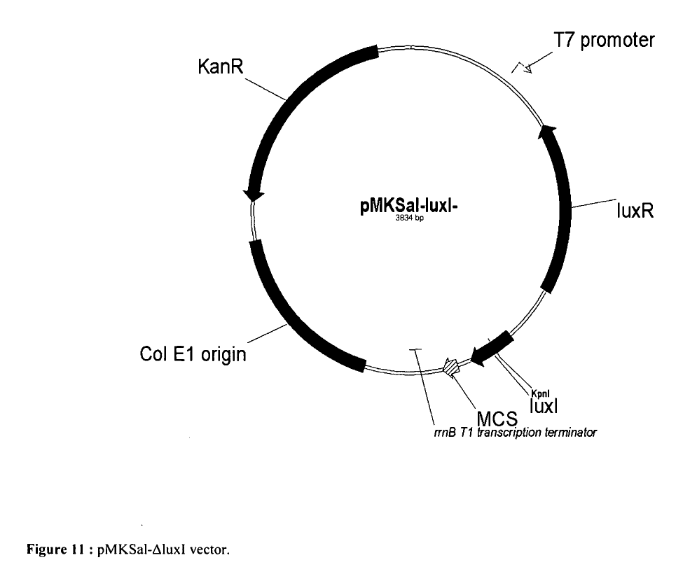

Construction of pMKSal-ΔluxI and pMKSal-ΔluxI-gfp and dosage of the autoinducer. The luxI gene was truncated after digestion of the pMKSaI plasmid by the KpnI restriction enzymes which were inserted when the luxI sequence was optimized. They cover 70% of the sequence, and the luxI gene with the KpnI fragment deleted is not functional. After digestion, the linear plasmid was auto-circularized and was called pMKSal-ΔluxI (FIG. 11).

The gfp gene was inserted in the MCS of pMKSal-ΔluxI and the new vector was called pMKSal-ΔluxI-gfp. This plasmid was used for a semi-quantitative dosage of the autoinducer in culture broth. The dosage of the autoinducer, here the 3OC6-HSL, is based on the fact that MM294.1/pMKSal-ΔluxI-gfp cannot produce a functional LuxI protein and, therefore, the autoinducer. Thus, production of Gfp protein will be correlated to the concentration of 3OC6HSL present in the supernatant of the sample analyzed.

MM294.1/pMKSal-ΔluxI-gfp strains were grown in YE3X at 25° C., then the replication was blocked by the addition of the inhibitor trimetroprime. The cells were resuspended in filtered supernatant of the culture to be tested. The presence of the autoinducer in the supernatant was monitored by the measuring fluorescence.

New auto-induction system pMKSa1-ΔluxI in the E. coli MM294.1::luxI strain. The auto-induction system comprises the luxI gene, which was integrated in the metE locus in E. coli 294.1 by homologous recombination; and the vector pMKSal-ΔluxI, into which the gene of interest can be cloned. The advantages of this system include a reduction of the gene dosage of luxI to one copy per cell; minimal expression of LuxI in pre-induced condition; a slow and more controllable accumulation of autoinducer; reaching of the critical concentration of autoinducer at a higher cell density than is obtained with a high gene dosage of luxI; and the ability to independently control the gene dosage of luxI and the gene dosage of the gene of interest.

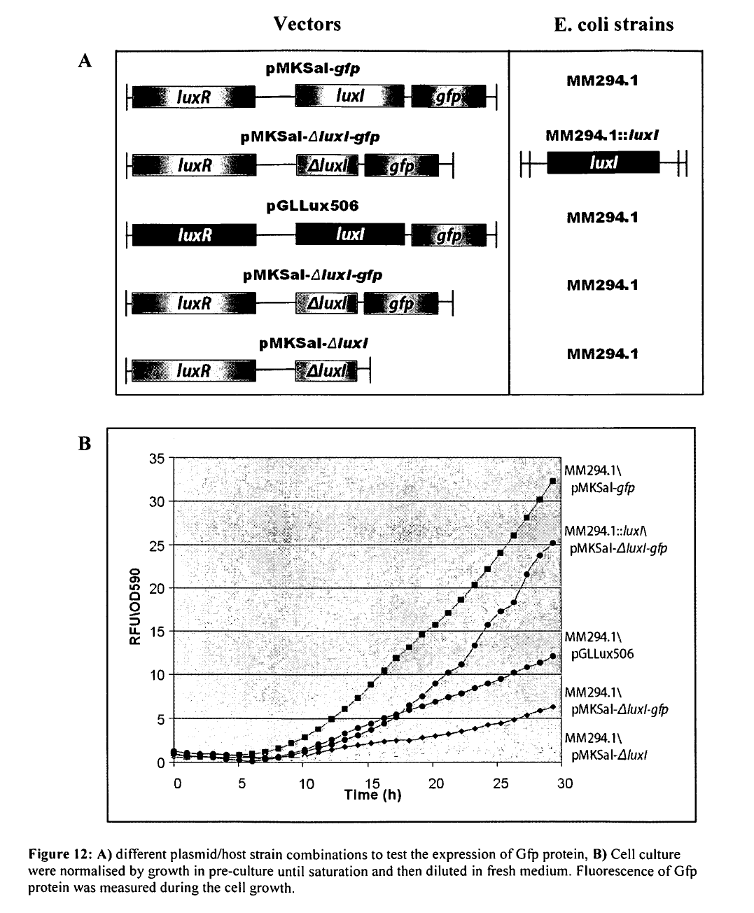

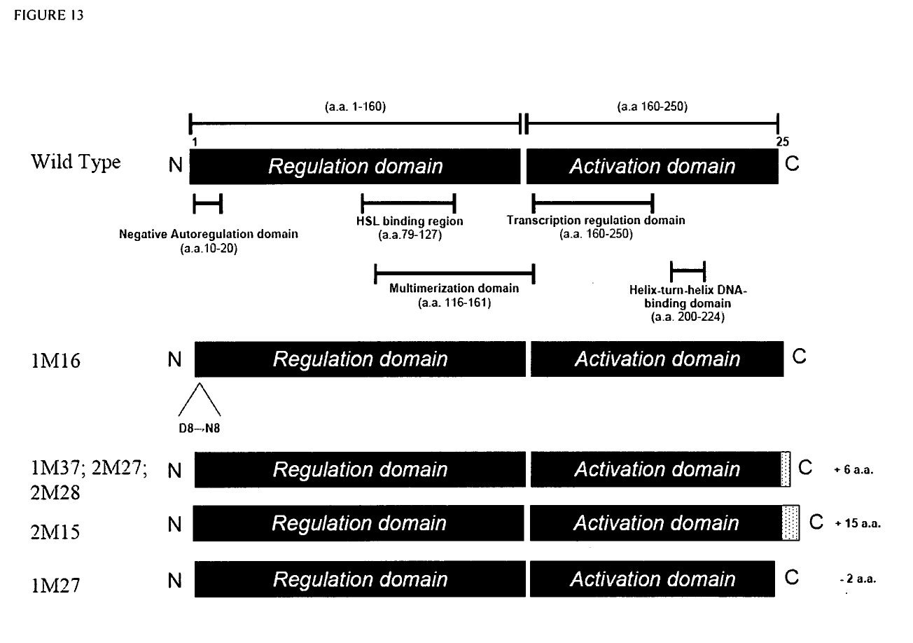

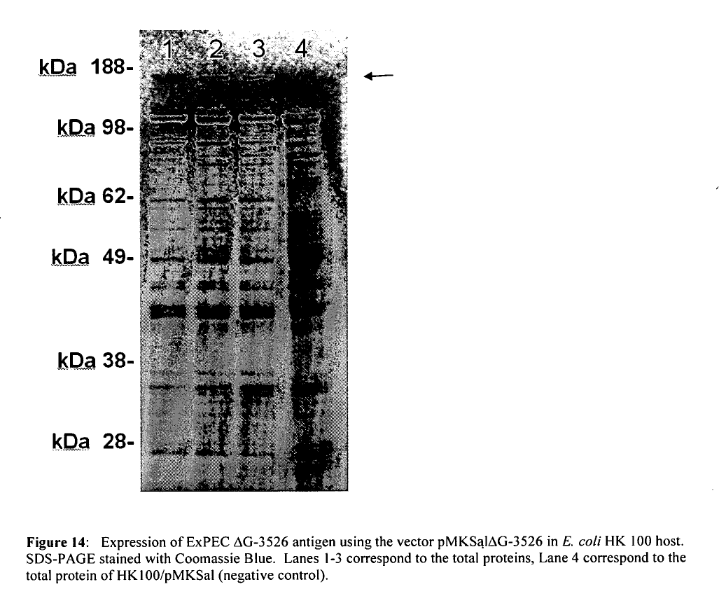

Several different vectors were tested in different host cells (FIG. 12A). The expression of the protein of interest, here the Gfp protein, was followed by measuring the fluorescence (FIG. 12B). MM294.1 comprising the control vector pMKSal-ΔluxI-GFP demonstrated a basal level of fluorescence. MM294.1 cells comprising the pMKSal-GFP vector produced GFP at a lower cellular density (OD 0.5-0.7 at 590 nm) compared to cells comprising the other vectors tested and expressed higher levels of GFP than control cells or cells comprising the vector pGLLux506. MM294.1 cells comprising the vector pGLLux506 express GFP at lower levels compared to MM294.1 cells comprising the vector pMKSal-GFP; in cells comprising the vector pGLLux506, expression of GFP was induced at a cellular density with an OD between 1.5-2.2 at 590 nm and the induction was gradual.

Host cells with only one copy of luxI per cell integrated (the MM294.1::luxI strain) and comprising the pMKSa1-ΔluxI-GFP plasmid are induced to produce GFP at a cellular density with an OD between 4.5 and 6 at 590 nm. The system is activated later compared to the other combinations tested, which is an advantage for large scale production of recombinant proteins.

### Example 8

### Synthesis of ExPEC ΔG-3526 Antigen

The ExPEC ΔG-3526 gene was cloned in the pMKSa1 plasmid. The new vector, called pMKSa1 ΔG-3526, was introduced in the HK 100 strain of E. coli. An overnight inoculum was diluted 1/100 to a final OD590 of 0.3 in 50 ml of HTMC medium (30 g/l Yeast Extract, 16 g/l K₂HPO₄, 4 g/l KH₂PO₄, 15 g/l Glycerol) with 30 mg/l Kanamycin and was grown at 27° C. Aliquots were taken at 17 h, 22 h, and 42 hours. Aliquots were loaded on SDS-PAGE gel and the gel was stained with Coomassie blue to assess the expression of the AG-3526 antigen. Expression of ΔG-3526 using this auto-inducer system is shown in FIG. 14. The AG-3526 protein has the sequence as described in SEQ ID NO: 152. The nucleic acide sequence which encodes the AG-3526 protein is described in SEQ ID NO: 151

1. An isolated mutant LuxR protein selected from the group consisting of (a) a mutant LuxR protein which has an extended C-terminal amino acid sequence relative to a wild type LuxR protein; (b) mutant LuxR proteins having a truncated C-terminal amino acid sequence and improved regulatory activity relative to a wild-type LuxR protein; (c) mutant LuxR proteins having a C-terminal amino acid sequence which is truncated by only 1, 2, 3, 4, 5, 6, 7, 8, or 9 amino acids in length relative to a wild type LuxR protein numbered according to SEQ ID NO:42; and (d) a mutant LuxR protein which comprises an amino acid alteration at one or more of amino acid positions 8-20, wherein the amino acid positions are numbered according to SEQ ID NO:42.

2. The isolated mutant LuxR protein of claim 1 wherein the C-terminal amino acid sequence of (a) is extended by between about 5 and about 20 amino acids.

3. The isolated mutant LuxR protein of claim 2 wherein the C-terminal amino acid sequence is extended by 6 amino acids or by 15 amino acids in length relative to the wild type LuxR protein.

4. The isolated mutant LuxR protein of claim 3 wherein the extended C-terminal amino acid sequence is VKYVSKA (amino acids 250-256 of SEQ ID NO:72) or VKYVSKAKGNSTTLD (amino acids 250-264 of SEQ ID NO:75).

5. The isolated mutant LuxR protein of claim 1, wherein the protein is selected from the group consisting of: mutant LuxR proteins having a truncated C-terminal amino acid sequence and improved regulatory activity relative to a wild-type LuxR protein; and mutant LuxR proteins having a C-terminal amino acid sequence which is truncated by only 1, 2, 3, 4, 5, 6, 7, 8, or 9 amino acids in length relative to a wild type LuxR protein numbered according to SEQ ID NO:42.

6. The isolated mutant LuxR protein of claim 5 wherein the C-terminal amino acid sequence is truncated by 2 amino acids.

7. The isolated mutant LuxR protein of claim 1 which comprises an amino acid alteration at one or more of amino acid positions 8-20, wherein the amino acid positions are numbered according to SEQ ID NO:42.

8. The isolated mutant LuxR protein of claim 7 which comprises an amino acid alteration at position D8.

9. The isolated mutant LuxR protein of claim 8 which comprises the amino acid sequence SEQ ID NO:73.

10. The isolated mutant LuxR protein of claim 1 which exhibits improved regulatory activity relative to a wild type LuxR protein.

11. An isolated nucleic acid molecule which encodes a protein selected from the group consisting of (a) the mutant LuxR protein of claim 1; (b) a V. fischeri Luxl protein wherein the nucleotide sequence is optimized for expression in E. coli; and (c) a V. fischeri LuxR protein, wherein the nucleotide sequence is optimized for expression in E. coli.

12. The isolated nucleic acid molecule of claim 11 wherein (a) comprises a nucleotide sequence selected from the group consisting of SEQ ID NOS: 144, 145, 146, 147, 148 and 149.

13. The isolated nucleic acid molecule of claim 11 comprising a nucleotide sequence which encodes a V. fischeri Luxl protein or a V. fischeri LuxR protein, wherein the nucleotide sequence is optimized for expression in E. coli.

14. The isolated nucleic acid molecule of claim 13 wherein the nucleotide sequence comprises a nucleotide sequence selected from the group consisting of SEQ ID NOS:78-97, 133, and 134.

15. An expression vector comprising the isolated nucleic acid molecule of claim 11.

16. An expression vector, comprising: a first gene operably linked to a first promoter, wherein the first gene is induced by a LuxR-type protein/autoinducer complex; and a second gene operably linked to a second promoter, wherein the second promoter is not induced by the LuxR-type protein/autoinducer complex and wherein expression of the second gene interferes with expression of the first gene.

17. The expression vector of claim 16 wherein the first gene encodes a LuxR-type protein.

18. The expression vector of claim 17 wherein the LuxR-type protein is LuxR or a mutant LuxR protein selected from the group consisting of (a) a mutant LuxR protein which has an extended C-terminal amino acid sequence relative to a wild type LuxR protein; (b) mutant LuxR proteins having a truncated C-terminal amino acid sequence and improved regulatory activity relative to a wild-type LuxR protein; (c) mutant LuxR proteins having a C-terminal amino acid sequence which is truncated by only 1, 2, 3, 4, 5, 6, 7, 8, or 9 amino acids in length relative to a wild type LuxR protein numbered according to SEQ ID NO:42; and (d) a mutant LuxR protein which comprises an amino acid alteration at one or more of amino acid positions 8-20, wherein the amino acid positions are numbered according to SEQ ID NO:42.

19. The expression vector of claim 16 wherein the first gene encodes a protein of interest.

20. The expression vector of claim 16 further comprising a third promoter operably linked to a third gene encoding a Luxl-type protein.

21. The expression vector of claim 20 wherein the third gene encodes Luxl.

22. The expression vector of claim 21 further comprising a fourth promoter operably linked to a fourth gene encoding a LuxR-type protein.

23. The expression vector of claim 22 wherein the fourth gene encodes LuxR or a mutant LuxR protein selected from the group consisting of (a) a mutant LuxR protein which has an extended C-terminal amino acid sequence relative to a wild type LuxR protein; (b) mutant LuxR proteins having a truncated C-terminal amino acid sequence and improved regulatory activity relative to a wild-type LuxR protein; (c) mutant LuxR proteins having a C-terminal amino acid sequence which is truncated by only 1, 2, 3, 4, 5, 6, 7, 8, or 9 amino acids in length relative to a wild type LuxR protein numbered according to SEQ ID NO:42; and (d) a mutant LuxR protein which comprises an amino acid alteration at one or more of amino acid positions 8-20, wherein the amino acid positions are numbered according to SEQ ID NO:42

24. The expression vector of claim 16 wherein at least one of the first, third, and fourth genes is optimized for expression in E. coli.

25. An expression vector, comprising: a first gene encoding a Luxl-type protein operably linked to a first promoter; a second gene encoding a LuxR-type protein operably linked to a second promoter; a third gene encoding a protein of interest operably linked to a third promoter which is induced by a LuxR-type protein/autoinducer complex; and a repressor gene operably linked to a fourth promoter which is inducible but which is not induced by the LuxR-type protein/autoinducer complex, wherein expression of the repressor gene interferes with expression of luxR.

26. The expression vector of claim 25 wherein the first gene encodes Luxl.

27. The expression vector of claim 25 wherein the second gene encodes LuxR or a mutant LuxR protein selected from the group consisting of (a) a mutant LuxR protein which has an extended C-terminal amino acid sequence relative to a wild type LuxR protein; (b) mutant LuxR proteins having a truncated C-terminal amino acid sequence and improved regulatory activity relative to a wild-type LuxR protein; (c) mutant LuxR proteins having a C-terminal amino acid sequence which is truncated by only 1, 2, 3, 4, 5, 6, 7, 8, or 9 amino acids in length relative to a wild type LuxR protein numbered according to SEQ ID NO:42; and (d) a mutant LuxR protein which comprises an amino acid alteration at one or more of amino acid positions 8-20, wherein the amino acid positions are numbered according to SEQ ID NO:42

28. The expression vector of claim 25 wherein at least one of the first and second genes is optimized for expression in E. coli.

29. An isolated host cell comprising an expression vector selected from the group consisting of (a) an expression vector comprising a nucleic acid molecule which encodes the mutant LuxR protein of claim 1; (b) an expression vector comprising a first gene operably linked to a first promoter, wherein the first gene is induced by a LuxR-type protein/autoinducer complex, and a second gene operably linked to a second promoter, wherein the second promoter is not induced by the LuxR-type protein/autoinducer complex and wherein expression of the second gene interferes with expression of the first gene; and (c) an expression vector comprising a first gene encoding a Luxl-type protein operably linked to a first promoter, a second gene encoding a LuxR-type protein operably linked to a second promoter, a third gene encoding a protein of interest operably linked to a third promoter which is induced by a LuxR-type protein/autoinducer complex, and a repressor gene operably linked to a fourth promoter which is inducible but which is not induced by the LuxR-type protein/autoinducer complex, wherein expression of the repressor gene interferes with expression of luxR.

30. An isolated host cell comprising a heterologous gene selected from the group consisting of a first gene encoding a Luxl-type protein and a second gene encoding a LuxR— type protein, wherein the heterologous gene is stably integrated into the genome of the isolated host cell.

31. The isolated host cell of claim 30 wherein the heterologous gene is the first gene and the first gene encodes Luxl.

32. The isolated host cell of claim 30 wherein the heterologous gene is the second gene and the second gene encodes LuxR or a mutant LuxR protein selected from the group consisting of (a) a mutant LuxR protein which has an extended C-terminal amino acid sequence relative to a wild type LuxR protein; (b) mutant LuxR proteins having a truncated C-terminal amino acid sequence and improved regulatory activity relative to a wild-type LuxR protein; (c) mutant LuxR proteins having a C-terminal amino acid sequence which is truncated by only 1, 2, 3, 4, 5, 6, 7, 8, or 9 amino acids in length relative to a wild type LuxR protein numbered according to SEQ ID NO:42; and (d) a mutant LuxR protein which comprises an amino acid alteration at one or more of amino acid positions 8-20, wherein the amino acid positions are numbered according to SEQ ID NO:42

33. The isolated host cell of claim 30 which comprises the first gene and further comprising a third gene encoding a LuxR-type protein.

34. The isolated host cell of claim 33 wherein the third gene is stably integrated into the genome of the host cell.

35. The isolated host cell of claim 33 wherein the third gene encoding the LuxR-type protein is in an expression vector.

36. The isolated host cell of claim 33 wherein the LuxR-type protein is selected from the group consisting of LuxR and a mutant LuxR protein selected from the group consisting of (a) a mutant LuxR protein which has an extended C-terminal amino acid sequence relative to a wild type LuxR protein; (b) mutant LuxR proteins having a truncated C-terminal amino acid sequence and improved regulatory activity relative to a wild-type LuxR protein; (c) mutant LuxR proteins having a C-terminal amino acid sequence which is truncated by only 1, 2, 3, 4, 5, 6, 7, 8, or 9 amino acids in length relative to a wild type LuxR protein numbered according to SEQ ID NO:42; and (d) a mutant LuxR protein which comprises an amino acid alteration at one or more of amino acid positions 8-20, wherein the amino acid positions are numbered according to SEQ ID NO:42

37. The isolated host cell of claim 30 further comprising an expression vector which comprises a gene of interest operably linked to an inducible promoter, wherein the inducible promoter is induced by the LuxR-type protein/autoinducer complex.

38. The isolated host cell of claim 30 wherein at least one of the first, second, and third genes is optimized for expression in E. coli.

39. An isolated host cell comprising: a heterologous gene encoding a LuxR-type protein; and an expression vector encoding a gene of interest operably linked to a promoter which is induced by a LuxR-type protein/autoinducer complex.

40. The isolated host cell of claim 39 wherein the heterologous gene is present in an expression vector.

41. The isolated host cell of claim 39 wherein the heterologous gene is stably integrated into the genome of the host cell.

42. The isolated host cell of claim 39 wherein the heterologous gene encodes LuxR or a mutant LuxR protein selected from the group consisting of (a) a mutant LuxR protein which has an extended C-terminal amino acid sequence relative to a wild type LuxR protein; (b) mutant LuxR proteins having a truncated C-terminal amino acid sequence and improved regulatory activity relative to a wild-type LuxR protein; (c) mutant LuxR proteins having a C-terminal amino acid sequence which is truncated by only 1, 2, 3, 4, 5, 6, 7, 8, or 9 amino acids in length relative to a wild type LuxR protein numbered according to SEQ ID NO:42; and (d) a mutant LuxR protein which comprises an amino acid alteration at one or more of amino acid positions 8-20, wherein the amino acid positions are numbered according to SEQ ID NO:42.

43. The isolated host cell of claim 29 which is an E. coli cell.

44. A process for expressing a gene of interest in a host cell, comprising: culturing the isolated host cell of either one of claims 30 or 39 under conditions which permit expression of the gene of interest.

45. The process of claim 44 further comprising: preparing an inoculum of a host cell comprising an expression vector which comprises a first gene operably linked to a first promoter, wherein the first gene is induced by a LuxR-type protein/autoinducer complex; and a second gene operably linked to a second promoter, wherein the second promoter is not induced by the LuxR-type protein/autoinducer complex and wherein expression of the second gene interferes with expression of the first gene; and using the inoculum to prepare a culture of the host cell.

46. The process of claim 44 further comprising purifying a recombinant protein expressed by the gene of interest.

47. The process of claim 46 further comprising formulating a pharmaceutical composition comprising the recombinant protein.

48. The process of claim 47 wherein the pharmaceutical composition is a vaccine composition.

49. A method of optimizing expression of V. fischeri; luxl or luxR genes, comprising: obtaining a nucleotide sequence encoding Luxl or LuxR; and modifying the polynucleotide sequence to optimize codon usage in E. coli.

50. A recombinant protein obtained by the process of claim 44.

51. The recombinant protein of claim 50 which is ExPEC ΔG-3526.

52. A pharmaceutical composition obtained by the method of claim 47.

53. The pharmaceutical composition of claim 51 which is a vaccine composition.

54. The isolated host cell of claim 30 which is an E. coli cell.

55. The isolated host cell of claim 39 which is an E. coli cell.
